(19) United States
(12) Patent Application Publication
(10) Pub. No.: US 2008/0242662 A1
(43) Pub. Date: Oct. 2, 2008

(54) Organic Compounds

(76) Inventors: Atsuko Nihonyanagi, Ibaraki (JP); Atsushi Toyao, Ibaraki (JP); Yuki Iwaki, Ibaraki (JP); Keiichi Masuya, Bottmingen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/915,673

(22) PCT Filed: May 29, 2006
(86) PCT No.: PCT/EP2006/005107

§ 371 Date: Nov. 27, 2007

(30) Foreign Application Priority Data
May 31, 2005 (GB) 0511063.0

(51) Int. Cl. A61K 31/495; C07D 241/04; C07D 403/12; A61K 31/538; A61P 43/00; A61K 31/496; C07D 265/36

(52) U.S. Cl. 514/230.5; 544/384; 544/373; 544/105; 514/255.01; 514/254.09

(57) ABSTRACT

The invention relates to substituted 3,4- or higher substituted piperazine compounds, the use thereof for the preparation of a pharmaceutical formulation for the treatment of a disease that depends on activity of renin; the use of a compound of that class in the treatment of a disease that depends on activity of renin; these compounds for use in the diagnostic and therapeutic treatment of a warm-blooded animal, especially for the treatment of a disease (=disorder) that depends on activity of renin; pharmaceutical formulations or products comprising said compounds, and/or a method of treatment comprising administering said compounds, a method for the manufacture of said compounds as well as novel intermediates, starting materials and/or partial steps for their synthesis. The compounds are especially of the formula I,

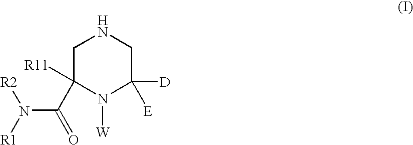

wherein R1, R2, R11, C, E and D are as defined in the specification.

The invention relates to substituted 3,4- or higher substituted piperazine compounds, the use thereof for the preparation of a pharmaceutical formulation for the treatment of a disease that depends on activity of renin; the use of a compound of that class in the treatment of a disease that depends on activity of renin; these compounds for use in the diagnostic and therapeutic treatment of a warm-blooded animal, especially for the treatment of a disease (=disorder) that depends on activity of renin; pharmaceutical formulations or products comprising said compounds, and/or a method of treatment comprising administering said compounds, a method for the manufacture of said compounds, as well as novel intermediates, starting materials and/or partial steps for their synthesis.

The invention especially relates to a compound of the formula I,

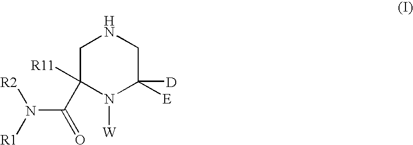

wherein

R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;

R2 is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;

W is a moiety selected from those of the formulae IA, IB and IC,

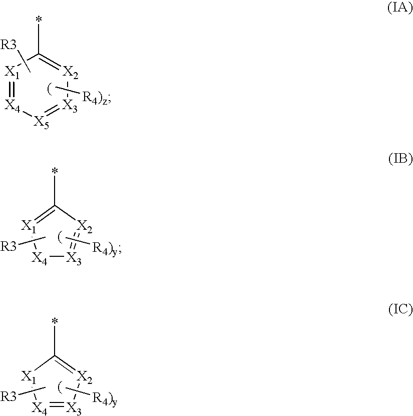

wherein the asterisk (*) denotes the position where the moiety W is bound to the 4-carbon in the piperidine ring in formula I, and wherein

X₁, X₂, X₃, X₄ and X₅ are independently selected from carbon and nitrogen, where X₄ in formula IB and X₁ in formula IC may have one of these meanings or further be selected from S and O, where carbon and nitrogen ring atoms can carry the required number of hydrogen or substituents R₃ or (if present within the limitations given below) R₄ to complete the number of bonds emerging from a ring carbon to four, from a ring nitrogen to three; with the proviso that in formula IA at least 2, preferably at least 3 of X₁ to X₅ are carbon and in formulae IB and IC at least one of X₁ to X₄ is carbon, preferably two of X₁ to X₄ are carbon;

y is 0, 1, 2 or 3;

z is 0, 1, 2, 3 or 4

(the obligatory moiety) R3 which can only be bound to any one of X₁, X₂, X₃ and X₄ (instead of a hydrogen in a fictive ring without R3 and replacing it) is unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro or cyano;

R4 (which is preferably bound to a ring atom other than that to which R₃ is bound) is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro and cyano;

each of D and E is hydrogen, or D and E together form oxo (═O); and

R11 is hydrogen, C₁-C₇-alkyl, halo-C₁-C₇-alkyl, cycloalkyl, halo-substituted cycloalkyl or cyano,

or a (preferably pharmaceutically acceptable) salt thereof.

The compounds of the present invention exhibit inhibitory activity on the natural enzyme renin. Thus, compounds of formula I may be employed for the treatment (this term also including prophylaxis) of one or more disorders or diseases selected from, inter alia, hypertention atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, especially as far as these diseases can be modulated (more especially beneficially influenced) by renin inhibition.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo. If not explicitly or implicitly stated otherwise, halo can also stand for more than one halogen substituent in moieties such as alkyl, alkanoyl and the like (e.g. in trifluoromethyl, trifluoroacetyl).

Unsubstituted or substituted alkyl is preferably C₁-C₂₀-alkyl, more preferably C₁-C₇-alkyl, that is straight-chained or branched (one or, if desired and possible, more times), and which is unsubstituted or substituted by one or more, e.g. up to three moieties selected from unsubstituted or substituted aryl or aryloxy with aryl in both cases as described below, especially phenyl, naphthyl, phenyloxy or naphthyloxy each of which is unsubstituted or substituted as described below for unsubstituted or substituted aryl, unsubstituted or substituted heterocyclyl as described below, especially pyrrolyl, furanyl, thienyl (=thiophenyl), thiazolyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydro-pyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl or benzo[1,2,5]oxadiazolyl each of which is unsubstituted or substituted as described below for unsubstituted or substituted heterocyclyl; unsubstituted or substituted cycloalkyl as described below, especially cyclopropyl, cyclobutyl, cyclopentyl or cyclohexyl each of which is unsubstituted or substituted as described below for unsubstituted or substituted cycloalkyl; halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkynylene, C₁-C₇-alkylenedioxy, sulfenyl (—S—OH), sulfinyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl (—S(O)₂OH), C₁-C₇-alkylsulfonyl (C₁-C₇-alkyl-SO₂—), phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl, N-mono or N,N-di-(C₁-C₇-alkyl, phenyl, naphthyl, phenyl-C₁-C₇-alkyl or naphthyl-C₁-C₇-alkyl)-aminosulfonyl and in position 2 or higher with regard to the binding carbon (as otherwise unsubstituted or substituted alkanoyl would result which falls under acyl) oxo.

Unsubstituted or substituted alkenyl preferably has 2 to 20 carbon atoms and includes one or more double bonds, and is more preferably C₂-C₇-alkenyl that is unsubstituted or substituted as described above for unsubstituted or substituted alkyl. Examples are vinyl or allyl.

Unsubstituted or substituted alkynyl preferably has 2 to 20 carbon atoms and includes one or more triple bonds, and is more preferably C₂-C₇-alkynyl that is unsubstituted or substituted as described above for unsubstituted or substituted alkyl. An example is prop-2-ynyl.

Unsubstituted or substituted aryl preferably is a mono- or bicyclic aryl moiety with 6 to 22 carbon atoms, especially phenyl (very preferred), or naphthyl (very preferred), and is unsubstituted or substituted by one or more, especially one to three, moieties, preferably independently selected from the group consisting of a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below; especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di-(C₁-C₇-alkyl)-amino, mono- di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N—) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N-C₁-C₇alkoxy-C₁-C₇-alkylcarbamoyl or N-mono- or N,N-di-(C₁-C₇-alkyl)-aminosulfonyl;

from C₂-C₇-alkenyl, C₂-C₇-alkynyl, phenyl, naphthyl, heterocyclyl, especially as defined below for heterocyclyl, preferably selected from pyrrolyl, furanyl, thienyl, thiazolyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydro-pyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N—) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N—) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxy-napthtyl-)aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N—) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl. Especially preferably aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇-alkoxy, hydroxy-C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carboxyl-C₁-C₇-alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano.

Unsubstituted or substituted heterocyclyl is a mono- or polycyclic, especially mono- or bicyclic, heterocyclic moiety with an unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen and sulfur (—S—, S(═O)— or S—(═O)₂—) which is unsubstituted or substituted by one or more, e.g. up to three, substitutents preferably independently selected from the substitutents mentioned above for aryl and from oxo (═O) and thioxo (═S). Preferably, unsubstituted or substituted heterocyclyl is selected from the following moieties:

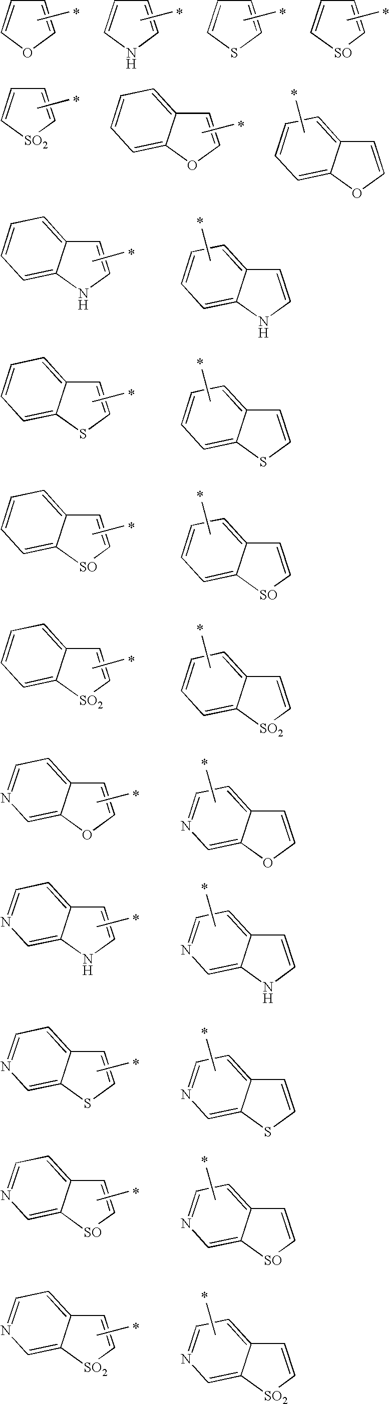

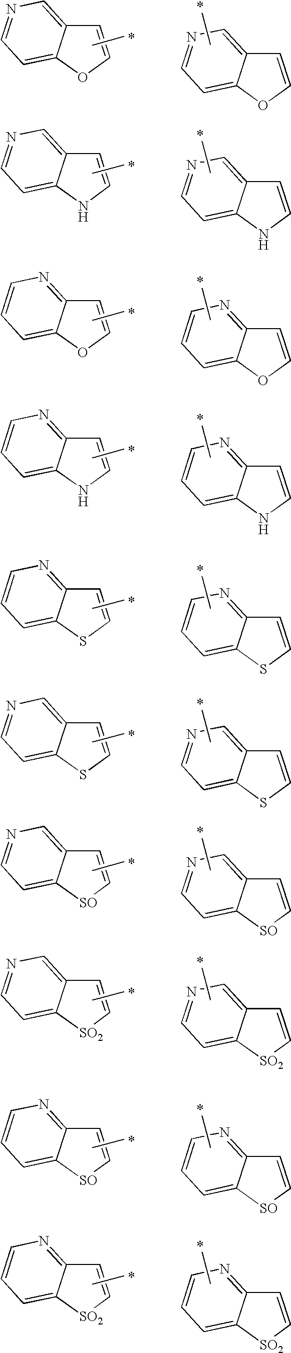

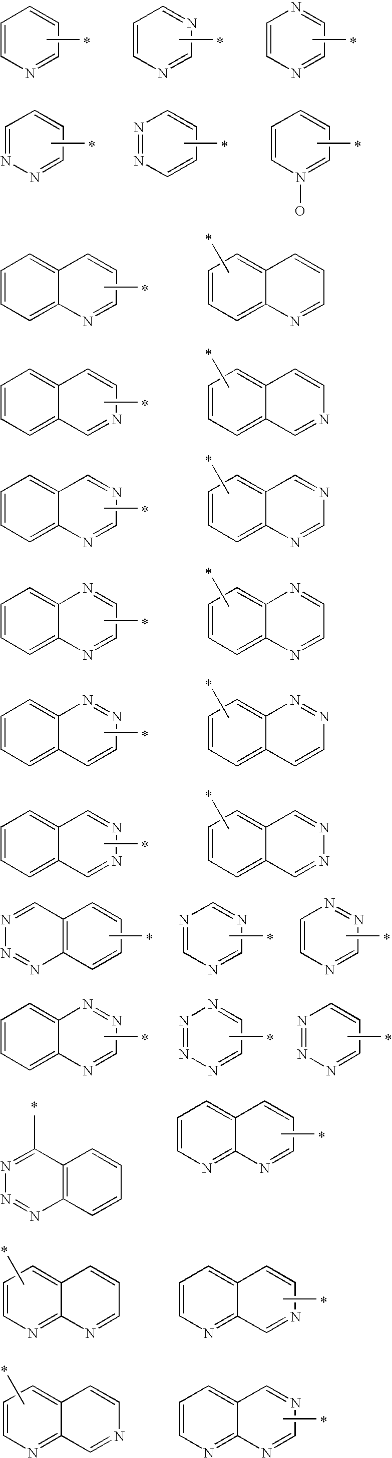

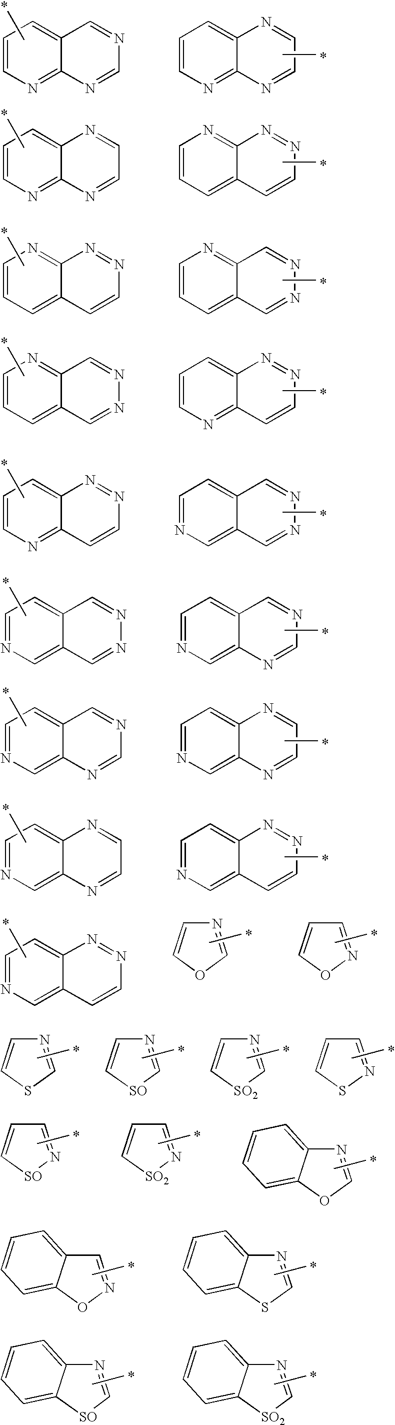

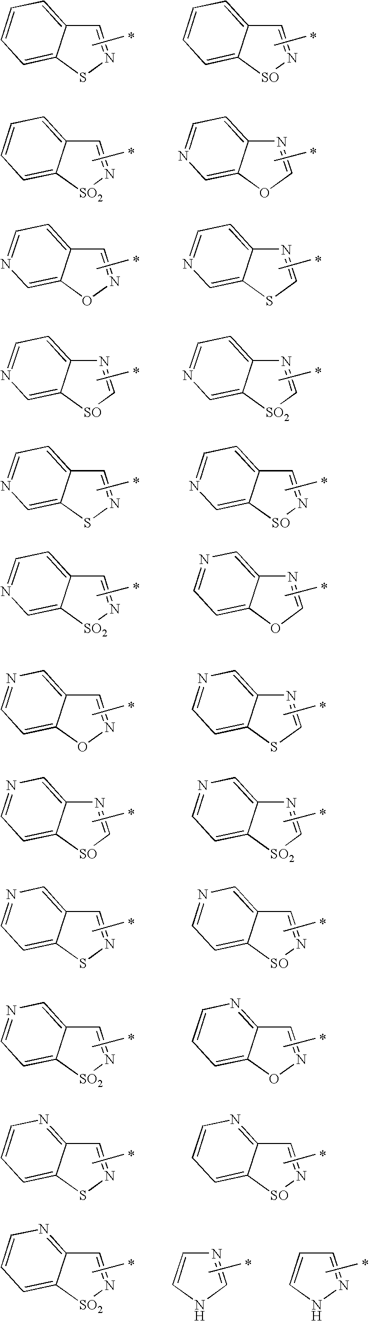

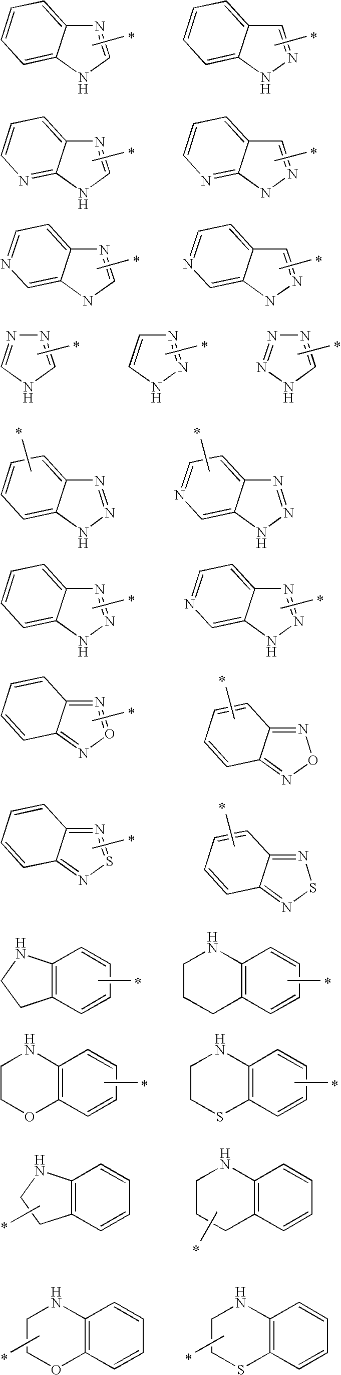

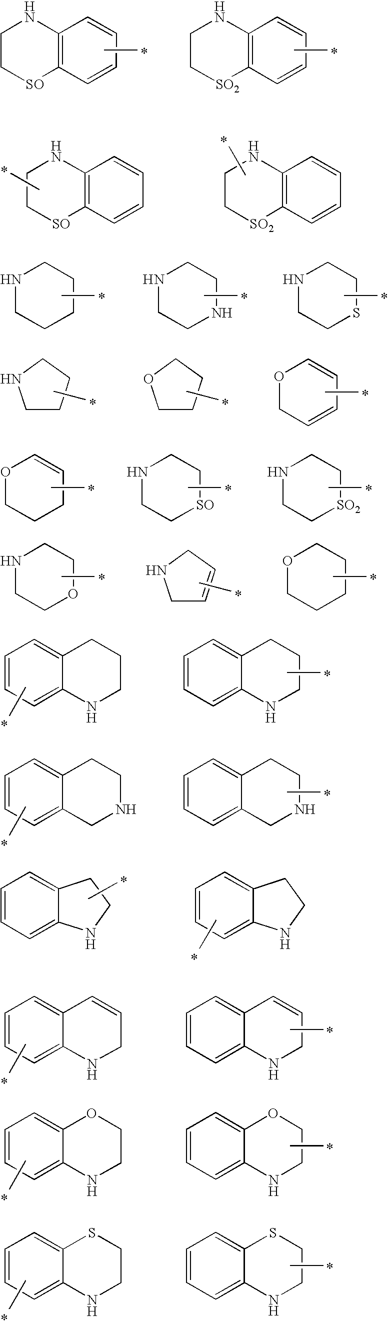

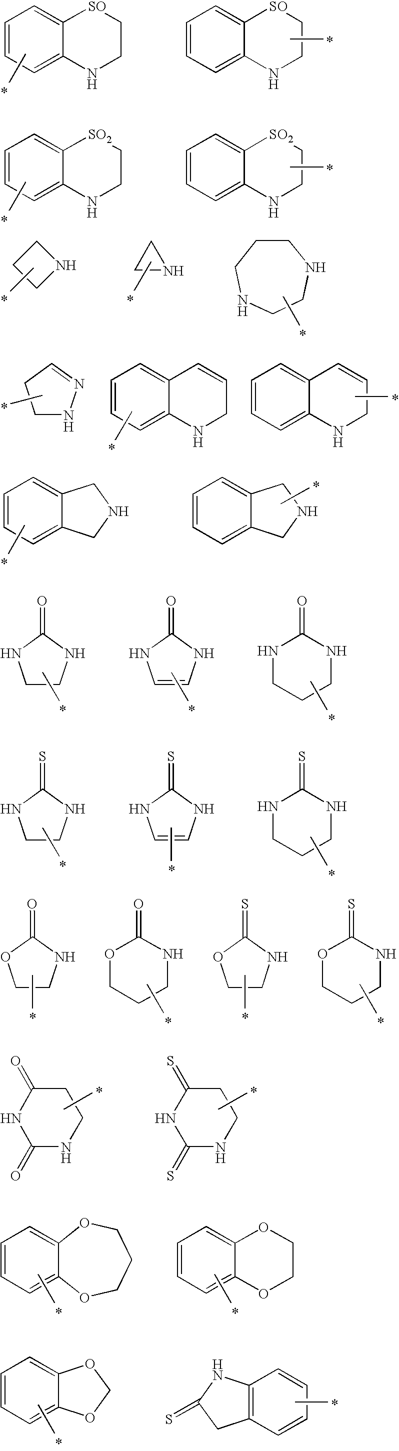

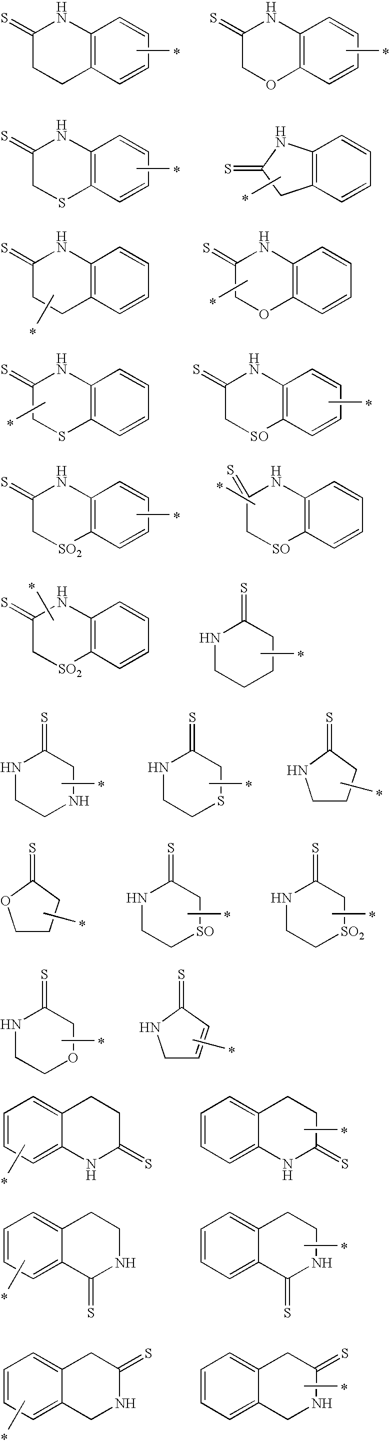

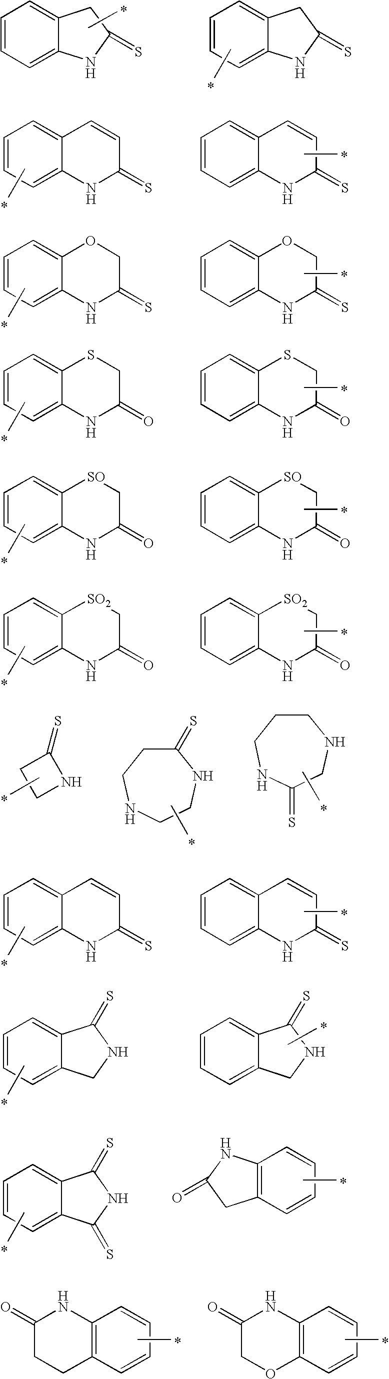

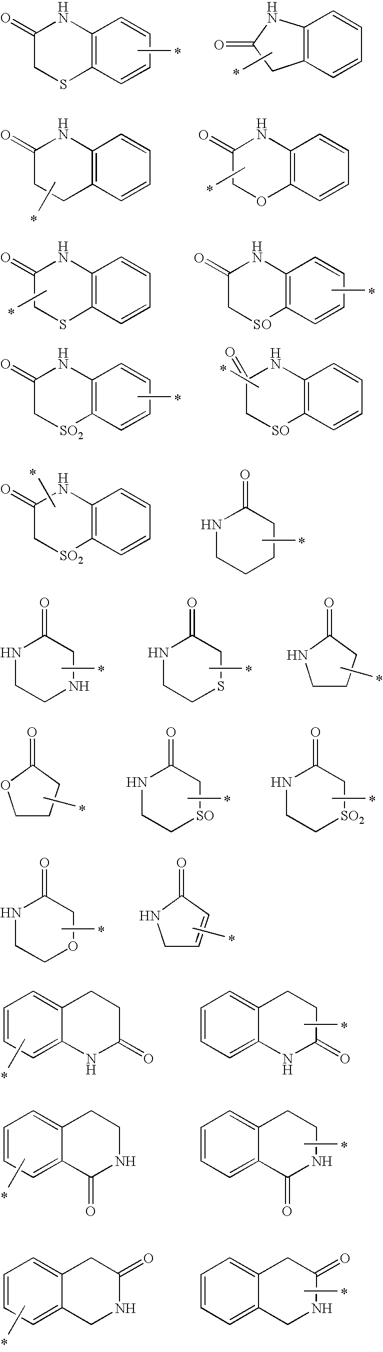

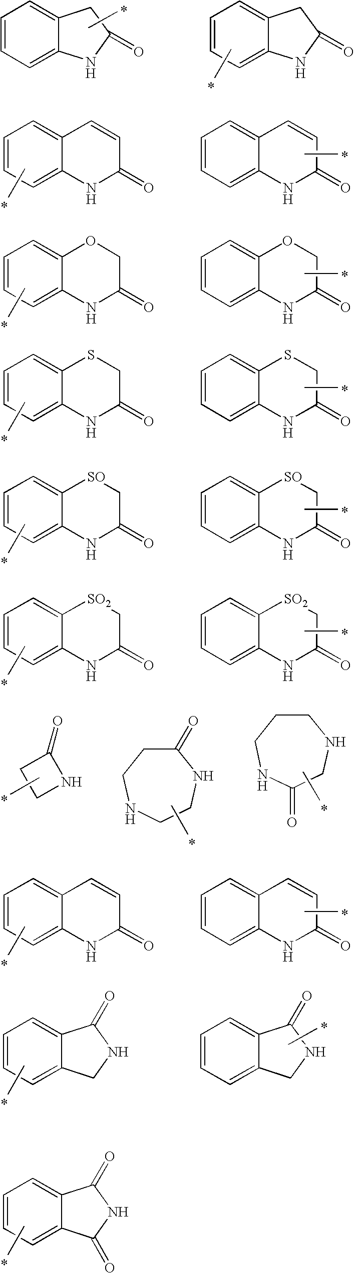

where in each case where an H is present bound to a ring atom the bond with the asterisk connecting the respective heterocyclyl moiety to the rest of the molecule the H may be replaced with said bond and if present one or more further H atoms bound to a ring atom may be replaced by one or more substituents as just described. Preferred as unsubstituted or substituted heterocyclyl are indolyl or 2H-1,4-benzoxazin-3(4H)-only, each of which is unsubstituted or substituted by one or mote, especially up to three substituents independently selected from the substituents mentioned for substituted aryl above.

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkynyl), and is unsubstituted or substituted by one or more, e.g. one to three substitutents preferably independently selected from those mentioned above as substituents for aryl. Preferred is cyclopropyl, cyclobutyl, cyclopentyl, cyclohexyl or cycloheptyl.

Acyl is preferably unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, or unsubstituted or substituted alkyloxycarbonyl or -oxysulfonyl, unsubstituted or substituted aryl-oxycarbonyl or -oxysulfonyl, unsubstituted or substituted heterocyclyloxycarbonyl or -oxysulfonyl, unsubstituted or substituted cycloalkyloxycarbonyl or -oxysulfonyl or N-mono- or N,N-di- (unsubstituted or substituted aryl, unsubstituted or substituted heterocyclyl, unsubstituted or substituted cycloalkyl or unsubstituted or substituted alkyl)-aminocarbonyl; wherein unsubstituted or substituted aryl, unsubstituted or substituted heterocyclyl, unsubstituted or substituted cycloalkyl and unsubstituted or substituted alkyl are preferably as described above. Preferred is C₁-C₇-alkanoyl, such as acetyl, 3,3-dimethyl-butyryl, 2,2-dimethyl-propionyl or 3,3-dimethyl-butyryl, unsubstituted or mono-, di- or tri-(halo and/or C₁-C₇-alkyl)-substituted benzoyl or naphthoyl, such as 4-methyl-benzoyl, C₃-C₈-cycloalkylcarbonyl, such as cyclobutylcarbonyl, unsubstituted or phenyl-substituted pyrrolidinylcarbonyl, especially phenyl-pyrrolidinocarbonyl, C₁-C₇-alkylsulfonyl, such as methylsulfonyl (=methanesulfonyl), (phenyl- or naphthyl)-C₁-C₇-alkylsulfonyl, such as phenylmethanesulfonyl, or (unsubstituted, or [C₁-C₇-alkyl-, phenyl-, halo-lower alkyl-, halo, oxo-C₁-C₇-alkyl-C₁-C₇-alkyloxy-, phenyl-C₁-C₇-alkoxy-, halo-C₁-C₇-alkyloxy-, phenoxy-, C₁-C₇-alkanoylamino-, cyano-, C₁-C₇-alkanoyl- and/or C₁-C₇-alkylsulfonyl-]substituted) (phenyl- or naphthyl)-sulfonyl, such as phenylsulfonyl (=benzenesulfonyl), naphthalene-1-sulfonyl, naphthalene-2-sulfonyl, toluene-4-sulfonyl, 4-isopropyl-benzenesulfonyl, biphenyl-4-sulfonyl, 2-trifluoromethyl-benzenesulfonyl, 4-chloro-benzenesulfonyl, 3-chloro-benzenesulfonyl, 2-chloro-benzenesulfonyl, 2,4-difluoro-benzenesulfonyl, 2,6-difluoro-benzenesulfonyl, 2,5-dichloro-benzenesulfonyl, 3,4-dichloro-benzenesulfonyl, 3,5-dichloro-benzenesulfonyl, 2,3-dichloro-benzenesulfonyl, 3-methoxy-benzenesulfonyl, 4-methoxy-benzenesulfonyl, 2,5-dimethoxy-benzenesulfonyl, 4-trifluoromethoxy-benzenesulfonyl, 2-benzyloxy-benzenesulfonyl, 3-trifluoromethyl-benzenesulfonyl, 4-phenoxy-benzenesulfonyl, 4-(2-oxo-propyl)-benzenesulfonyl, 4-acetylamino-benzenesulfonyl, 4-cyano-benzenesulfonyl, 2-cyano-benzenesulfonyl, 3-cyano-benzenesulfonyl, 3-acetyl-benzenesulfonyl or 4-methanesulfonyl-benzenesulfonyl, halo-thiophene-2-sulfonyl, such as 5-chloro-thiophene-2-sulfonyl, quinoline-sulfonyl, such as quinoline-8-sulfonyl, (C₁-C₇-alkanoylamino and/or C₁-C₇-alkyl)-substituted thiazol-sulfonyl, such as 2-acetylamino-4-methyl-thiazole-5-sulfonyl, (halo and/or C₁-C₇-alkyl)-substituted pyrazolesulfonyl, such as 5-chloro-1,3-dimethyl-1H-pyrazole4-sulfonyl, pyridine-sulfonyl, such as pyridine-3-sulfonyl, or N-mono- or N,N-di-(C₁-C₇-alkyl, (unsubstituted or halo-substituted) phenyl or naphthyl, phenyl-C₁-C₇-alkyl, naphthyl-C₁-C₇-alkyl or C₃-C₈-cycloalkyl)-aminocarbonyl, such as N-tert-butyl-aminocarbonyl, (3-chloro-phenyl)-aminocarbonyl, N-benzyl-aminocarbonyl, N-cyclohexyl-aminocarbonyl, C₁-C₇-alkylaminocarbonyl or phenyl-C₁-C₇alkylaminocarbonyl, or (C₁-C₇-alkyl, phenyl, naphthyl, phenyl-C₁-C₇-alkyl and/or napthyl-C₁-C₇-alkyl)-oxycarbonyl, e.g. C₁-C₇-alkoxycarbonyl, such as tert-butyloxycarbonyl or isobutyloxycarbonyl, or phenyl-C₁-C₇-alkyloxycarbonyl.

“-Oxycarbonyl-” means —O—C(═O)—, “aminocarbonyl” means in the case of mono-substitution —NH—C(═O)—, in the case of double substitution also the second hydrogen is replaced by the corresponding moiety. For example, C₁-C₇-alkoxycarbonyl is C₁-C₇-alkyl-O—C(═O)—.

That R3 can only be bound to any one of X₁, X₂, X₃ and X₄ means that this moiety cannot be bound in p-position of ring IA.

Etherified or esterified hydroxy is especially hydroxy that is esterified with acyl as defined above, especially in C₁-C₇-alkanoyloxy; or preferably etherified with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkyloxy, especially with a substituent selected from C₁-C₇-alkoxy; from phenyl, tetrazolyl, tetrahydrofuran-onyl, oxetidinyl, 3-(C₁-C₇-alkyl)-oxetidinyl, pyridyl or 2H,3H-1,4-benzodioxinyl, each of which is unsubstituted or substituted by one or more, preferably up to three, e.g. 1 or two substituents independently selected from C₁-C₇-alkyl, hydroxy, C₁-C₇-alkoxy, phenyloxy wherein phenyl is unsubstituted or substituted, preferably up to three times, by C₁-C₇-alkoxy and/or by halo, phenyl-C₁-C₇-alkoxy wherein phenyl is unsubstituted or substituted, preferably up to three times, by C₁-C₇-alkoxy and/or halo; halo, amino, N-mono- or N,N-di(C₁-C₇-alkyl, phenyl, naphthyl, phenyl-C₁-C₇-alkyl or naphthyl-C₁-C₇-alkyl)amino, C₁-C₇-alkanoylamino, carboxy, C₁-C₇-alkoxycarbonyl, phenyl- or naphthyl-C₁-C₇-alkoxycarbonyl, N-mono- or N,N-di(C₁-C₇-alkyl, phenyl, naphthyl, phenyl-C₁-C₇-alkyl or naphthyl-C₁-C₇-alkyl)-aminocarbonyl, morpholino, morpholino-C₁-C₇-alkoxy, pyridyl-C₁-C₇-alkoxy, pyrazolyl, 4-C₁-C₇-alkylpiperidin-1-yl and cyano; or selected from morpholino;

or unsubstituted or substituted aryloxy with unsubstituted or substituted aryl as described above, especially phenyloxy with phenyl that is unsubstituted or substituted, preferably up to three times, by C₁-C₇-alkoxy and/or by halo; or

unsubstituted or substituted heterocyclyloxy with unsubstituted or substituted heterocyclyl as described above, preferably tetrahydropyranyloxy.

Substituted mercapto can be mercapto that is thioesterified with acyl as defined above, especially with lower alkanoyloxy; or preferably thioetherified with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylthio or unsubstituted or substituted arylthio with unsubstituted or substituted C₁-C₇-alkyl or aryl as just described for the corresponding moieties under etherified hydroxy.

Substituted sulfinyl or sulfonyl can be substituted with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylsulfinyl or -sulfonyl or unsubstituted or substituted arylsulfinyl or -sulfonyl with unsubstituted or substituted C₁-C₇-alkyl or aryl as just described for the corresponding moieties under etherified hydroxy.

In mono- or di-substituted amino, amino is preferably substituted by one or more substituents selected from one acyl, especially C₁-C₇-alkanoyl, phenylcarbonyl (=benzoyl), C₁-C₇₋ -alkylsulfonyl or phenylsulfonyl wherein phenyl is unsubstituted or substituted by one to 3 C₁-C₇-alkyl groups, and from one or two moieties selected from alkyl, alkenyl, alkynyl, aryl, heterocyclyl and cycloalkyl each of which is unsubstituted or substituted and is preferably as described above for the corresponding unsubstituted or substituted moieties. Preferred is C₁-C₇-alkanoylamino, mono- or di-(phenyl, naphthyl, C₁-C₇-alkoxy-phenyl, C₁-C₇-alkoxynaphthyl, naphthyl-C₁-C₇-alkyl or phenyl-C₁-C₇-alkyl)-carbonylamino (e.g. 4-methoxybenzoylamino), mono- or di-(C₁-C₇-alkyl and/or C₁-C₇-alkoxy-C₁-C₇-alkyl)-amino or mono- or di-(phenyl, naphthyl, C₁-C₇-alkoxy-phenyl, C₁-C₇-alkoxynaphthyl, phenyl-C₁-C₇-alkyl, naphthyl-C₁-C₇-alkyl, C₁-C₇-alkoxy-naphthyl-C₁-C₇-alkyl or C₁-C₇-alkoxy-phenyl-C₁-C₇-alkyl)-amino.

Esterified carboxy is preferably alkyloxycarbonyl, aryloxycarbonyl, heterocyclyloxycarbonyl or cycloalkyloxycarbonyl, wherein alkyl, aryl, heterocyclyl and cycloalkyl are unsubstituted or substituted and the corresponding moieties and their substituents are preferably as described above. Preferred is C₁-C₇-alkoxycarbonyl, phenyl-C₁-C₇-alkyloxycarbonyl, phenoxycarbonyl or naphthoxycarbonyl.

In amidated carboxy, the amino part bound to the carbonyl in the amido function (A₂N—C(═O)—) wherein each A is independently of the other hydrogen or an amino substituent) is unsubstituted or substituted as described for substituted amino, but preferably without acyl as amino substituent. Preferred is mono- or di-(C₁-C₇-alkyl and/or C₁-C₇-alkoxy-C₁-C₇alkyl)-aminocarbonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminocarbonyl.

In substituted sulfamoyl, the amino part bound to the sulfonyl in the sulfamoyl function (A₂N—S(═O)₂—) wherein each A is independently of the other hydrogen or an amino substituent) is unsubstituted or substituted as described for substituted amino, but preferably without acyl as amino substituent. Preferred is mono- or di-(C₁-C₇-alkyl and/or C₁-C₇-alkoxy-C₁-C₇alkyl)-aminosulfonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminosulfonyl.

Unsubstituted or substituted C₁-C₇-alkyl, unsubstituted or substituted C₂-C₇-alkenyl and unsubstituted or substituted C₂-C₇-alkynyl and their substituents are defined as above under the corresponding (un)substituted alkyl, (un)substituted alkynyl and (un)substituted alkynyl moieties but with the given number of carbon atoms in the alkyl, alkenyl or alkynyl moieties.

In halo-substituted cycloalkyl R11, cycloalkyl is preferably as defined above.

The following preferred embodiments of the moieties and symbols in formula I can be employed independently of each other to replace more general definitions and thus to define specially preferred embodiments of the invention, where the remaining definitions can be kept broad as defined in embodiments of the inventions defined above of below.

R1 is preferably hydrogen, C₁-C₇-alkyl, C₃-C₈-cycloalkyl or C₃-C₈-cycloalkyl-C₁-C₇-alkyl, more preferably hydrogen, ethyl or cyclopropyl.

R2 is preferably phenyl, phenyl-C₁-C₇-alkyl, naphthyl, naphthyl-C₁-C₇-alkyl, indolyl, indolyl-C₁-C₇-alkyl, 2H-1,4-benzoxazin-3(4H)-onyl or 2H-1,4-benzoxazin-3(4H)-onyl-C₁-C₇-alkyl, wherein each phenyl, naphthyl, indolyl or 2H-1,4-benzoxazin-3(4H)-onyl is unsubstituted or preferably substituted by one ot more, especially up to three, e.g. two, moieties independently selected from C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy, C₁-C₇-alkoxy-C₁-C₇-alkoxy and halo, more preferably R2 is 3-(3-methoxypropoxy)-4-methoxy-phenyl, 3-(2-methoxyethyl)-4-methoxy-phenyl, 3-(3-methoxypropoxy)-4-methyl-phenyl, 3-(2-methoxyethyl)-4-methyl-phenyl, 2-(2,3-dimethyl-phenyl)-methyl, 3-(3-methoxy-propoxy-methyl)-5-methoxy-phenylmethyl, 3-(2-methoxy-ethoxy-methyl)-5-methoxy-phenylmethyl, 3-(3-methoxy-propoxy)-5-methoxy-phenylmethyl, 3-(2-methoxy-ethoxy)-5-methoxy-phenylmethyl, 1-(3-methoxy-propyl)-indol-3-yl-methyl, 1-(2-methoxy-ethyl)-indol-3-yl-methyl, 5-fluoro-1-(3-methoxy-propyl)-indol-3-yl-methyl, 5-fluoro-1-(2-methoxy-ethyl)-indol-3-yl-methyl, 6-fluoro-1-(3-methoxy-propyl)-indol-3-yl-methyl, 6-fluoro-1-(2-methoxy-ethyl)-indol-3-yl-methyl, 4-(3-methoxypropyl)-2H-1,4-benzoxazin-3(4H)-on-6-yl or 4-(2-methoxyethyl)-2H-1,4-benzoxazin-3(4H)-on-6-yl.

W is preferably a moiety of the formula IA wherein each of X₁, X₂, X₃, X₄, X₄ and X₅ is CH or a moiety of the formula IC wherein X₁ is S, X₂ is N, X₃ is CH and X₄ is CH, and R3 is selected from the group consisting of phenyl, hydroxy, phenyloxy-C₁-C₇-alkyl and phenyl-C₁-C₇-alkoxy, where each phenyl mentioned in the present definition of W so far is unsubstituted or substituted by one or more moieties independently selected from hydroxy, C₁-C₇-alkoxy, carboxy-C₁-C₇-alkoxy, C₁-C₇-alkoxycarbonyl-C₁-C₇-alkoxy and phenyl- or naphthyl-C₁-C₇-alkoxycarbonyl-C₁-C₇-alkoxy, more preferably, W is 3-phenyl-phenyl, 3-hydroxyphenyl, 3-(4-hydroyphenyl)-phenyl, 3- or 2-[(3,5-dimethoxy-phenyl)-methoxy]-phenyl, 3-[(4-carboxyl-methyloxy)-phenyl]-phenyl or 4-phenyl-thiazol-2-yl.

Each of y and z is preferably 1 or more preferably 0 (zero).

Each of D and E is hydrogen or D and E together form oxo. In one embodiment, D and E are both hydrogen. In another embodiment E and D form oxo.

R11 is preferably hydrogen.

Preferably, compounds of the present application have the formula

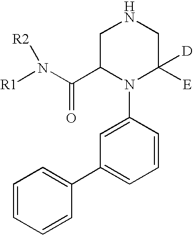

wherein R1, R2, R3, D and E are as defined herein inparticular with respect to the preferred embodiments, or a (preferably pharmaceutically acceptable) salt thereof.

In all definitions above and below the person having skill in the art will, without undue experimentation or considerations, be able to recognize which are relevant (e.g. those that if present provide compounds that are sufficiently stable for the manufacture of pharmaceuticals, e.g. having a half-life of more than 30 seconds) and thus are preferably encompassed by the present claims and that only chemically feasible bonds and substitutions (e.g. in the case of double or triple bonds, hydrogen carrying amino or hydroxy groups and the like) are encompassed, as well as tautomeric forms where present, especially in equilibrium. For example, preferably, for reasons of stability or chemical feasibility, directly vicinal atoms in chains preferably are not selected from oxy plus oxy, thio plus oxy, oxy plus thio or thio plus thio, except where ring systems or the like are present that are sufficiently stable. Substitutents binding via an O (e.g. in C₁-C₇-alkoxy) or S that is part of them are preferably not bound to nitrogen e.g. in rings.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds”, “starting materials” and “intermediates” hereinbefore and hereinafter, especially to the compound(s) of the formula I or their precursors, is to be understood as referring also to one or more salts thereof or a mixture of a corresponding free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, starting materials, intermediates, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural (for example also different configuration isomers of the same compound, e.g. enantiomers in racemates or the like) or preferably the singular (“one”).

The compounds of the present invention can possess one or more asymmetric centers depending on the choice of the substituents. The preferred absolute configurations are as indicated herein specifically. However, any possible isolated or pure diastereoisomers, enantiomers or geometric enantiomers, and mixtures thereof, e.g., mixtures of enantiomers, such as racemates, are encompassed by the present invention.

As described above, the compounds of the present invention are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, Alzheimer's disease, dementia, anxiety states and cognitive disorders, and the like, especially where inhibition of (especially inappropriate) renin activity is required.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration and/or the like) and/or leads to or supports a renin dependent disease or disorder as mentioned above and below, e.g. by too high renin activity. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore “dependent” can be read as “dependent inter alia”, (especially in cases where a disease or disorder is really exclusively dependent only on renin) preferably as “dependent mainly”, more preferably as “dependent essentially only”. A disease dependent on (especially inappropriate) activity of renin may also be one that simply responds to modulation of renin activity, especially responding in a beneficial way (e.g. lowering the blood pressure) in case of renin inhibition.

Where a disease or disorder dependent on (=that “depends on”, “depending”) (especially inappropriate) activity of a renin is mentioned (such in the definition of “use” in the following paragraph and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural renin and/or one or more altered or mutated forms thereof.

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more diseases or disorders mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate, and each of the more specific definitions, independent of any others, may be introduced independently of or together with one or more other more specific definitions for other more general expressions or symbols.

In a first preferred embodiment, the invention relates to a compound of the formula I wherein

R1 is hydrogen, C₁-C₇-alkyl, C₃-C₈-cycloalkyl or C₃-C₈-cycloalkyl-C₁-C₇-alkyl;

R2 is phenyl, phenyl-C₁-C₇-alkyl, naphthyl, naphthyl-C₁-C₇-alkyl, indolyl, indolyl-C₁-C₇-alkyl, 2H-1,4-benzoxazin-3(4H)-onyl or 2H-1,4-benzoxazin-3(4H)-onyl-C₁-C₇-alkyl, wherein each phenyl, naphthyl, indolyl or 2H-1,4-benzoxazin-3(4H)-onyl is unsubstituted or preferably substituted by one or more, especially up to three, e.g. two, moieties independently selected from C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy, C₁-C₇-alkoxy-C₁-C₇-alkoxy and halo;

W is a moiety of the formula IA wherein each of X₁, X₂, X₃, X₄, X₄ and X₅ is CH or a moiety of the formula IC wherein X₁ is S, X₂ is N, X₃ is CH and X₄ is CH, and R3 which is bound to any one of X₁, X₂, X₃ or X₄ in formula IA or to any one of X₃ and X₄ in formula IC is selected from the group consisting of phenyl, hydroxy, phenyloxy-C₁-C₇-alkyl and phenyl-C₁-C₇-alkoxy, where each phenyl mentioned in the present definition of W so far is unsubstituted or substituted by one or more moieties independently selected from hydroxy, C₁-C₇-alkoxy, carboxy-C₁-C₇-alkoxy, C₁-C₇-alkoxycarbonyl-C₁-C₇-alkoxy and phenyl- or naphthyl-C₁-C₇-alkoxycarbonyl-C₁-C₇-alkoxy;

each of y and z is 0 (zero) (that is, no R4 is present to replace a H);

each of D and E is hydrogen of D and E together form oxo; and

R11 is hydrogen;

or a (preferably pharmaceutically acceptable) salt thereof.

More preferably, the invention relates to a compound of the formula I wherein

R1 is hydrogen, ethyl or cyclopropyl;

R2 is 3-(3-methoxypropoxy)-4-methoxy-phenyl, 3-(2-methoxyethyl)-4-methoxy-phenyl, 3-(3-methoxypropoxy)-4-methyl-phenyl, 3-(2-methoxyethyl)-4-methyl-phenyl, 2-(2,3-dimethyl-phenyl)-methyl, 3-(3-methoxy-propoxy-methyl)-5-methoxy-phenylmethyl, 3-(2-methoxy-ethoxy-methyl)-5-methoxy-phenylmethyl, 3-(3-methoxy-propoxy)-5-methoxy-phenylmethyl, 3-(2-methoxy-ethoxy)-5-methoxy-phenylmethyl, 1-(3-methoxy-propyl)-indol-3-yl-methyl, 1-(2-methoxy-ethyl)-indol-3-yl-methyl, 5-fluoro-1-(3-methoxy-propyl)-indol-3-yl-methyl, 5-fluoro-1-(2-methoxy-ethyl)-indol-3-yl-methyl, 6-fluoro-1-(3-methoxy-propyl)-indol-3-yl-methyl, 6-fluoro-1-(2-methoxy-ethyl)-indol-3-yl-methyl, 4-(3-methoxypropyl)-2H-1,4-benzoxazin-3(4H)-on-6-yl or 4-(2-methoxyethyl)-2H-1,4-benzoxazin-3(4H)-on-6-yl,

W is 3-phenyl-phenyl, 3-hydroxyphenyl, 3-(4-hydroyphenyl)-phenyl, 3- or 2-[(3,5-dimethoxy-phenyl)-methoxy]-phenyl, 3-[(4-carboxyl-methyloxy)-phenyl]-phenyl or 4-phenyl-thiazol-2-yl,

each of D and E is hydrogen of D and E together form oxo; and

R11 is hydrogen;

or a (preferably pharmaceutically acceptable) salt thereof.

The invention especially relates to a compound of the formula I with the configuration shown in the following formula (A),

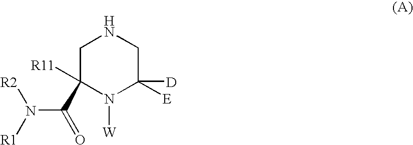

wherein R1, R2, R11, W, D and E are as defined for a compound of the formula I, or a (preferably pharmaceutically acceptable) salt thereof.

Alternatively, the invention especially relates to a compound of the formula I shown in the following formula (B)

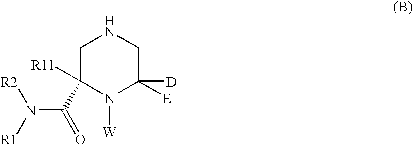

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as the use thereof.

Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by

reacting a carbonic acid of the formula II,

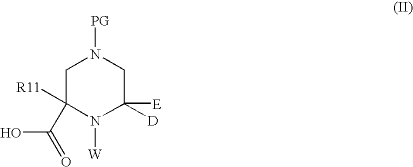

or a reactive derivative thereof, wherein PG is a protecting group and W and R11 are as defined for a compound of the formula I, with an amino compound of the formula III,

R1-NH—R2 (III)

wherein R1 and R2 are as defined for a compound of the formula I,

and, if desired, subsequent to this condensation reaction, converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;

where in any of the starting materials of the formula II and/or III, in addition to specific protecting groups mentioned, further protecting groups may be present, and any protecting groups are removed at an appropriate stage (especially before or after a reaction mentioned under “if desired”) in order to obtain a corresponding compound of the formula I, or a salt thereof.

Preferred Reaction Conditions

The preferred reaction conditions for the reactions mentioned above, as well as for the transformations and conversions, are as follows (or analogous to methods used in the Examples or as described there):

The condensation of a carbonic acid of the formula II, or a reactive derivative thereof, preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also and preferably be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine, diisopropylethylamine (DIEA) or N-methylmorpholine and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′, N′-tetramethyluronium tetrafluoroborate (TBTU); (benzotriazol-1-yloxy)-tripyrrolidinophosphonium-hexafluorophosphate (PyBOP), 1-(3-dimethylaminopropyl)-3-ethylcarbodiimide hydrochloride/hydroxybenzotriazole or/1-hydroxy-7-azabenzotriazole (EDC/HOBT or EDC/HOAt), HOAt alone, 4-(4,6-dimethoxy-1,3,5-triazin-2-yl)-4-methylmorpholinium chloride (DMT-MM) or with 1-chloro-2-methyl-propenyl)-dimethylamine (=1-chloro-N,N,2-trimethyl-1-propenyl-amine). For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction may preferably carried out under an inert gas, e.g. nitrogen or argon.

In order to obtain a compound of the formula I if no further conversion in the protected state is desired, the subsequent removal of a protecting group, e.g. PG, such as tert-butoxycarbonyl, benzyl, 9H-fluoren-9-ylmethoxycarbonyl or 2-(trimethylsilyl)-ethoxycarbonyl, takes place under standard conditions, see also the literature mentioned below under General Process Conditions. For example, tert-butoxycarbonyl is removed in the presence of an acid, e.g. a hydrohalic acid, such as HCl, in an appropriate solvent, e.g. an ether, such as dioxane, or an alcohol, e.g. isopropanol, at customary temperatures, e.g. at room temperature, the removal of benzyl can be achieved e.g. by reaction with ethylchloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., or by removal by means of trimethylsilyl trifluoroacetate in the presence of a tertiary nitrogen base, such as 2,6-lutidine, in an appropriate solvent, such as a halogenated hydrocarbon, e.g. methylene chloride; the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetonitrile, preferably at elevated temperatures, e.g. under reflux conditions, and the removal of a 9H-fluoren-9-yl-methoxycarbonyl protecting group can be achieved in the presence of a secondary amine, especially piperidine, in an appropriate solvent, e.g. a halogenated hydrocarbons, such as methylene chloride, at preferred temperatures between 0 and 50° C., e.g. at about room temperature.

Optional Reactions and Conversions

A compound of the formula I, or a protected form thereof directly obtained according to any one of the preceding procedures (meaning that, if conversion is desired, a removal of protecting groups is not required in the above-mentioned condensation reaction or after introducing protecting groups anew) which are included subsequently as starting materials for conversions as well even if not mentioned specifically, can be converted into different compounds of the formula I according to known procedures, where required or desired after removal of protecting groups.

Where R1 is hydrogen in a compound of the formula I, this can be converted into the corresponding compound wherein R1 has a meaning other than hydrogen given for compounds of the formula I by reaction with a compound of the formula IV,

R1*-Q (IV)

wherein R1* is defined as R1 in a compound of the formula I other than hydrogen and Q is a leaving group (e.g. preferably selected from halo, e.g. chloro, from unsubstituted or substituted aryl-sulfonyloxy, such as toluolsulfonyloxy, from unsubstituted or substituted alkylsulfonyloxy, such as methylsulfonyloxy or trifluoromethylsulfonyloxy, with the reaction allowed to take place e.g. in the presence of a base, such as an alkali metal salt of a weaker acid, e.g. an alkali metal carbonate and/or an alkali metal hydrogencarbonate, such as sodium or potassium carbonate and/or sodium or potassium hydrogencarbonate (NaHCO₃ or KHCO₃) in an appropriate solvent, e.g. dioxane and/or H₂O, at preferred temperatures between −20 and 50° C., e.g. at −5 to 30° C.), or wherein Q is —CHO (so that the compound of the formula IV is an aldehyde) and then R1* is the complementary moiety for a moiety R1 that includes a methylene group (resulting in a group R1 of the formula R1*-CH₂—) e.g. under reductive amination conditions as follows: The reaction preferably takes place under customary conditions for reductive amination, e.g. in the presence of an appropriate hydrogenation agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature.

Hydroxy substituents, e.g. as substitutents R3 of moieties of the formulae IA, IB or IC, can be transformed into unsubstituted or substituted alkoxy or unsubstituted or substituted aryl, e.g. by alkylation or arylation reaction with the corresponding unsubstituted or substituted alkyl- or arylhalogenide, e.g. its bromide or iodide, in the presence of a base, e.g. potassium carbonate or an alkaline metal hydride, such as sodium hydride, in an appropriate solvent, e.g. N,N-dimethylformamide, e.g. at preferred temperatures between 0 and 50° C.

Hydroxy substituents, e.g. as substitutents R3 of moieties of the formulae IA, IB or IC, can be transformed into unsubstituted or substituted aryl, such as hydroxyphenyl, by first transforming the OH group into a leaving group, e.g. by reaction with trifluoromethane sulfonic anhydride to the trifluoromethanesulfonyloxy group, e.g. in the presence of a tertiary amine, such as diisopropyl-ethylamine, in an appropriate solvent, such as methylene chloride, preferably at low temperatures, e.g. from −80 to 0° C., such as at −78° C.; followed by reaction with an unsubstituted or substituted aryl-B(OH)₂ compound e.g. in the presence or a base, such as potassium phosphate, in an appropriate solvent, such as dioxane, in the presence of a catalyst, especially Pd(PPh₃)₄, at temperatures e.g. in the range from 20 to 80° C., e.g. at about 60° C.

Carboxy substitutents can be converted into esterified carboxy by reaction with corresponding alcohols, e.g. C₁-C₇-alkanols, or into amidated carboxy by reaction with corresponding amines, e.g. under condensation conditions analogous to those described above under the condensation reaction between a compound of the formula II and a compound of the formula III.

Esterified carboxy substituents can be converted into free carboxy by hydrolysis, e.g. in the presence of a base, such as potassium hydroxide, in an appropriate solvent, e.g. tetrahydrofurane, preferably at elevated temperatures, e.g. from 50° C. to the reflux temperature of the reaction mixture.

In some cases, the conversions preferably take place with compounds of the formula I in protected form; the subsequent removal of protecting group can be achieved as described above for the condensation reaction between a compound of the formula II and a compound of the formula III and below under “General Process Conditions”, yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers or enantiomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-)crystallization, and the like. Some possible methods that can also be used with other compounds analogously can be found in the Examples.

Starting Materials

In the subsequent description of starting materials (which term also includes intermediates) and their synthesis, R1, R2, R3, R4, X₁, X₂, X₃, X₄, R11, D, E, y, z and PG have the meanings given above or in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, from participating in a reaction, employing protecting groups, methods for their introduction and their removal are as described above or below, e.g. in the references mentioned under “General Process Conditions”. The person skilled in the art will readily be able to decide whether and which protecting groups are useful or required and at which stage it is appropriate to introduce, exchange and/or remove protecting groups.

A compound of the formula II can be prepared e.g. from a compound of the formula V,

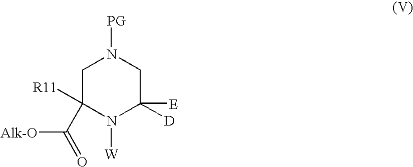

wherein Alk is the moiety of an alcohol, e.g. methyl or ethyl, and PG is a protecting group, especially tert-butoxycarbonyl, by hydrolysis, e.g. in the presence of a base, such as potassium or sodium hydroxide, in an appropriate solvent, e.g. aqueous methanol or ethanol or tetrahydrofurane, or a mixture thereof, at elevated temperatures, e.g. under reflux conditions.

A compound of the formula V wherein each of D and E are hydrogen can be obtained from the corresponding oxo compound of the formula V wherein D and E together are oxo by reduction e.g. with an appropriate complex hydride, such as BH₃/tetrahydrofurane, in an appropriate solvent, such as tetrahydrofurane, e.g. at temperatures in the range from 0 to 50° C.

A compound of the formula V wherein D and E together are oxo can be obtained from a compound of the formula V*

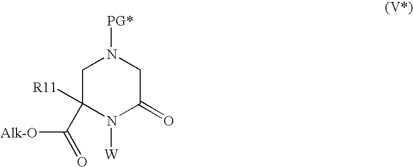

wherein PG* is a protecting group different from PG in a compound of the formula V, especially benzyl, by first removing the group PG*, e.g. in the case of PG*=benzyl reacting it with chloroformic acid-1-chloro-methylester in an appropriate solvent, e.g. 1,2-dichloroethane, at preferably elevated temperatures, e.g. from 80° C. to the reflux temperature to give the deprotected compound; into the latter then a protecting group PG can be introduced, e.g. tert-butoxycarbonyl by reaction with tert-butoxycarbonic anhydride in the presence of a mild base, e.g. an alkali metal hydrogencarbonate, such as sodium hydrogencarbonate, in an appropriate solvent, such as dichloromethane, at temperatures e.g. from 0 to 50° C.

A compound of the formula V* can preferably be prepared from a compound of the formula VI,

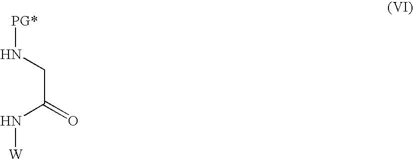

wherein PG* is as defined for a compound of the formula V* e.g. by first reacting it with with methyl-α-chloroacrylate in the presence of a tertiary base, such as diisopropylethylamine, in an appropriate solvent, such as acetonitrile, e.g. at elevated temperatures in the range from 30 to 80° C., resulting in a compound of the formula VII;

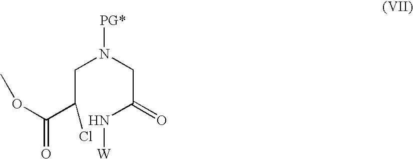

wherein PG* is as defined for a compound of the formula V*, which can then be reacted in the presence of a sufficiently strong base, especially an alkali metal hydride, in an appropriate solvent, e.g. dimethylformamide and/or tetrahydrofurane, e.g. at temperatures from 0 to 50° C. to the corresponding compound of the formula V*.

A compound of the formula VI can, for example, be prepared by reacting a compound of the formula VIII,

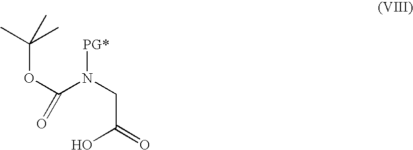

wherein PG* is as defined for a compound of the formula V*, first with chloroformic acid ethylester in an appropriate solvent, e.g. tetrahydrofurane, in the presence of a tertiary nitrogen base, such as triethylamine, e.g. at temperatures from 0 to 50° C., then reacting the product with a strong base, such as an alkali metal hydride, e.g. sodium hydride, with a compound of the formula IX,

W—NH₂ (IX)

wherein W is as defined for a compound of the formula I, e.g. in an appropriate solvent such as tetrahydrofurane, for example at temperatures from 0 to 50° C., and then removing the tert-butoxycarbonyl protecting group present at the same nitrogen as PG*, e.g. by treatment with an acid, such as HCL, in an appropriate solvent, such as dioxane, e.g. at temperatures from 0 to 50° C.

According to an alternative method, a compound of the formula VI can be obtained by reacting a compound of the formula X,

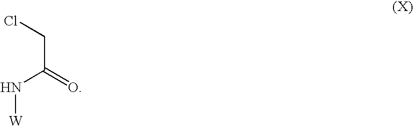

(obtainable e.g. in analogy to known procedures, see e.g. Eg. J. Pharm. Sci. 32, 251-261 (1991)) with a compound of the formula XI,

PG*-NH₂ (XI)

wherein PG* is as defined for a compound of the formula V*, e.g. in the presence of a base, such as potassium carbonate, and an alkali metal iodide, especially potassium iodide, in an appropriate solvent, e.g. acetonitrile, at temperatures in the range e.g. from 0 to 50° C.

A compound of the formula III may, for example, be prepared by reacting an amino compound of the formula XII,

R1-NH₂ (XII)

wherein R1 is as defined for a compound of the formula I with an aldehyde of the formula XIII,

R2*-CHO (XIII)

wherein R2* is the complementary moiety for a moiety R2 that includes a methylene group (resulting in a group R2 of the formula R2*-CH₂—) e.g. under reaction conditions as follows: The corresponding reaction (reductive amination) can take under customary conditions, e.g. in the presence of an appropriate hydrogenation agent, such as hydrogen in the presence of a catalyst or a complex hydride, e.g. sodium triacetoxyborohydride or sodium cyanoborohydride, in an appropriate solvent, such as a halogenated hydrocarbon, e.g. methylene chloride or 1,2,-dichloroethane, and/or an alcohol, such as methanol, and optionally a carbonic acid, e.g. acetic acid, at preferred temperatures between −10° C. and 50° C., e.g. from 0° C. to room temperature.

In a compound of the formula XIII wherein R2* is a heterocyclyl comprising an NH in the ring, such as indolyl, the H can be replaced with unsubstituted or substituted alkyl by reaction with a corresponding (unsubstituted or substituted alkyl)-halogenide or -tosylate (toluolsulfonyloxy-group comprising), e.g. in the presence of a base, such as sodium or potassium hydride, a corresponding halogenide salt, e.g. potassium iodide, and an appropriate solvent, e.g. N,N-dimethyl-formamide or the like, at temperatures e.g. in the range from −10 to 50° C. C, e.g. from 0 to 25° C., giving the corresponding compound of the formula X with an N-bound unsubstituted or substituted alkyl.

A compound of the formula XIII can be obtained by reducing a corresponding hydroxymethylene precursor of the formula XIV,

R2*-CH₂—OH (XIV)

under appropriate conditions, e.g. in the presence of manganese dioxide and an appropriate solvent, e.g. an ester, such as ethyl acetate, at appropriate temperatures, e.g. in the range from 10 to 80° C., e.g. at about room temperature to about 60° C.

A hydroxymethylene compound of the formula XIV can, for example, be obtained from a carbonic acid ester of the formula XV,

R2*-COOAlk (XV)

wherein R2* is as mentioned for a compound of the formula XIII and Alk is the moiety of an alcohol, e.g. of methyl or ethyl, by reduction under appropriate conditions, e.g. in the presence of an appropriate complex hydride, such as lithium aluminium hydride, in a customary solvent, such as a cyclic ether, e.g. tetrahydrofurane, at temperatures e.g. from −30 to 50° C., e.g. at about 0° C.

In a compound of the formula XIII or XV wherein R2* is substituted aryl carrying a hydroxymethylene group (and possibly other substituents), the hydroxymethylene group can be reacted with an unsubstituted or substituted alkyl-tosylate, e.g. a C₁-C₇-alkoxy-C₁-C₇-to-sylate, e.g. in the presence of a base, such as sodium or potassium hydride or an alkali metahl carbonate, such as potassium carbonate, a corresponding halogenide salt, e.g. potassium iodide, and an appropriate solvent, e.g. N,N-dimethyl-formamide or the like, at temperatures e.g. in the range from −10 to 50° C. C, e.g. from 0 to 25° C., giving the corresponding compound of the formula X or XIII carrying an (further unsubstituted or substituted) aryl with a corresponding unsubstituted or substituted alkyl-oxy-methyl substituent, e.g. C₁-C₇-alkoxy-C₁-C₇-alkoxy-methyl.

A compound of the formula III wherein R2 is aryl or heterocyclyl each of which is unsubstituted or substituted can be prepared by reducing a nitro compound of the formula XVI,

R2-NO₂ (XVI)

wherein R2 is aryl or heterocyclyl each of which is unsubstituted or substituted, e.g. in the presence of a non-noble metal, such as iron, in the presence of an acid, e.g. hydrochloric acid, in an appropriate solvent, e.g. an alcohol, such as ethanol, for example at elevated temperatures, e.g. from 40 to 80° C. or to the reflux temperature. This leads to a compound of the formula XVII,

R2-NH₂ (XVII)

wherein R2 is as just described which can then be protected by introducing an amino protecting group e.g. tert-butoxycarbonyl, under standard conditions, e.g. tert-butoxycarbonyl by reaction with tert-butoxycarbonic anhydride in the presence of a mild base, e.g. an alkali metal hydrogencarbonate, such as sodium hydrogencarbonate, in an appropriate solvent, such as dichloromethane, at temperatures e.g. from 0 to 50° C. This results in a compound of the formula XVIII,

R2-NH—PGₓ (XVIII)

wherein R2 is as described for a compound of the formula XVI and PGₓ is the amino protecting group introduced. This latter compound can then be reacted with a compound of the formula XIX,

R1-L (XIX)

wherein L is a leaving group, e.g. halo, such as bromo or iodo, in the presence of a strong base, such as an alkali metal hydride, in an appropriate solvent, such as tetrahydrofurane, e.g. at temperatures from 0 to 50° C.

Removal of the protecting group, e.g. by an acid such as hydrochloric acid in an appropriate solvent such as dioxane at temperatures e.g. from 0 to 50° C., of the product thus obtainable then leads to the corresponding compound of the formula III.

In a compound of the formula XVI wherein R2 is a heterocyclyl comprising an NH in the ring, such as indolyl or 2H-1,4-benzoxazin-3(4H)onyl, the H can be replaced with unsubstituted or substituted alkyl by reaction with a corresponding (unsubstituted or substituted alkyl)-halogenide or -tosylate (toluolsulfonyloxy-group comprising), e.g. in the presence of a base, such as sodium or potassium hydride, a corresponding halogenide salt, e.g. potassium iodide, and an appropriate solvent, e.g. N,N-dimethyl-formamide or the like, at temperatures e.g. in the range from −10 to 50° C. C, e.g. from 0 to 25° C., giving the corresponding compound of the formula XVI with an N-bound unsubstituted or substituted alkyl.

For introduction of moieties R11 other than hydrogen, a compound of the formula VI can be treated with a strong base to remove the hydrogen to be substituted by R11, such as lithium hexamethyldisialazide (LHMDS) or lithium diisopropylamide in tetrahydrofuran, for example at low temperatures, e.g. from −100 to −50° C., such as at −78° C., and then reacted with a C₁-C₇-alkylhalogenide, a cycloalkylhalogenide, a halo-C₁-C₇-alkyltosylate or a halo-cyclo-alkyltosylate to introduce the corresponding moieties C₁-C₇-alkyl, halo-C₁-C₇-alkyl, cycloalkyl or halo-substituted cycloalkyl.

The transformation reactions mentioned above for (unprotected or protected) compounds of the formula I, especially with regard to the conversion of R3=OH into unsubstituted or substituted aryloxy or unsubstituted or substituted alkyloxy, can also take place at any earlier stage where a group W carrying a hydroxy R3 (which can first be protected e.g. as methoxymethyl which protecting group can be removed at an appropriate stage as known from the literature, e.g. under conditions described in the examples).

Other starting materials, such as also the starting materials of the formula II, III; IV, V, VI, VII, VIII, XIX, X, XI, XII, XIII, XIV, XV, XVI, XVII, XVIII or XIX, as far as not already mentioned, are known in the art, can be prepared according to methods that are known in the art and/or are commercially available, or they can be prepared according to methods analogously to those mentioned in the examples.

General Process Conditions

The following applies in general (where possible) to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the processes in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the processes of the present invention those starting materials are preferably used which result in compounds of formula I described as being preferred. Special preference is given to reaction conditions that are identical or analogous to those mentioned in the Examples. The invention relates also to novel starting compounds and intermediates described herein, especially those leading to novel compounds of the formula I or compounds of the formula I mentioned as preferred herein.

Pharmaceutical Use, Pharmaceutical Preparations and Methods

As described above, the compounds of the formula I are inhibitors of renin activity and, thus, may be of use for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like. Hypertension, at least as one component of the disease to be treated, is especially preferred, meaning that hypertension alone or in combination with one or more (especially of the mentioned) other diseases may be treated (prophylactically and/or therapeutically).

The present invention further provides pharmaceutical compositions comprising a therapeutically effective amount of a pharmacologically active compound of the formula I, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders and the like. Especially preferred is a disease which comprises hypertension, more especially hypertension itself, where treatment with a pharmaceutical composition or the use of a compound of the formula I for its synthesis is useful prophylactically and/or (preferably) therapeutically.

Thus, the pharmacologically active compounds of the formula I may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and or polyvinylpyrrolidone; if desired

d) disintegrants, e.g., starches, agar, alginic acid or its sodium salt, or effervescent mixtures; and/or

e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;

c) anti-obesity agents such as orlistat; and

d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the figures 1 to 7, which are herein incorporated by reference. A compound of the formula I may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical products or compositions comprising a therapeutically effective amount of a compound of the formula I alone or in combination with a therapeutically effective amount of another therapeutic agent, preferably selected from anti-diabetics, hypolipidemic agents, anti-obesity agents and anti-hypertensive agents, most preferably from antidiabetics, anti-hypertensive agents and hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material.

The present invention further provides a method for the prevention and/or treatment of conditions mediated by (especially inappropriate) renin activity, which comprises administering a therapeutically effective amount of a compound of the formula I to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a pharmaceutical product comprising a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity, especially one or more of the specific diseases mentioned above.

Finally, the present invention provides a method or use which comprises administering a compound of formula I in combination with a therapeutically effective amount of an antidiabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC50 values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 20 μM.

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC50 values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 20 μM.

In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably can show IC₅₀ values in the range from 1 nM to 20 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably show IC₅₀ values in the range from 1 nM to 20 μM.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

Compounds of the formula I can be tested in vivo in primates as described in the literature (see for example by Schnell CR et al. Measurement of blood pressure and heart rate by telemetry in conscious, unrestrained marmosets. Am J Physiol 264 (Heart Circ Physiol 33). 1993: 1509-1516; or Schnell CR et al. Measurement of blood pressure, heart rate, body temperature, ECG and activity by telemetry in conscious, unrestrained marmosets. Proceedings of the fifth FELASA symposium: Welfare and Science. Eds BRIGHTON. 1993.

The following Examples, while in addition representing preferred embodiments of the invention, serve to illustrate the invention without limiting its scope.

The following Examples, while in addition representing preferred embodiments of the invention, serve to illustrate the invention without limiting its scope.

Abbreviations
Ac acetyl
aq. aqueous
Boc tert-butoxycarbonyl
Brine saturated sodium chloride solution
Celite trademark of Celite Corp. for filtering aid based on kieselguhr
conc. Concentrated
DCE 1,2-dichloroethane
DCM dichloromethane
DEAD diethyl azodicarboxylate
DIBAL diisobutylaluminum hydride
dppf 1,1′-Bis(diphenylphosphino)ferrocene
DIEA N,N-diisopropylethylamine
DMF N,N-dimethylformamide
DMSO dimethylsulfoxide
DMT-MM 4-(4,6-dimethoxy-1,3,5-triazin-2-yl)-4-methylmorpholinium chloride
EDC 1-(3-Dimethylaminopropyl)-3-ethylcarbodiimide hydrochloride
ES-MS electrospray mass spectrometry
Et ethyl
EtOAc ethyl acetate
h hour(s)
HMPA hexamethylphosphoramide
HOAt 1-hydroxy-7-azabenzotriazole
HPLC high-pressure liquid chromatography
HyFlo diatomaceous earth based filtering aid
IPr isopropyl
LAH lithium aluminium hydride
LDA lithium diisopropylamide
mCPBA 3-chloroperbenzoic acid
Me methyl
min minute(s)
mL milliliter(s)
MOMCl methoxymethyl chloride
MS Mass Spectrometry
MsCl Methylsulfonylchlorid
nBuLi n-butyllithium
n-Hex n-hexyl
NaOMe sodium methoxylate
NMP 1-methyl-2-pyrrolidinone
NMR nuclear magnetic resonance
Ph phenyl
RT room temperature
TBTU O-(benzotriazol-1-yl)-N,N,N′,N′-tetramethylammonium tetrafluoroborate
TFA trifluoroacetic acid
Tf₂O trifluoromethanesulfonic anhydride
THF tetrahydrofurane
TMS trimethylsilyl
TMSOTf trifluoromethanesulfonic acid trimethylsilyl ester
WSCD =EDC
t_{Ret} HPLC retention time in min determined by HPLC condition

Synthesis

Flash chromatography is performed by using silica gel (Merck; 40-63 μm). For thin layer chromatography, pre-coated silica gel (Merck 60 F254; Merck KGaA, Darmstadt, Germany)) plates are used. ¹NMR measurements are performed on a Bruker DXR 400 spectrometer using tetramethylsilane as internal standard. Chemical shifts (δ) are expressed in ppm downfield from tetramethylsilane. Electrospray mass spectra are obtained with a Fisons Instruments VG Platform II. Commercially available solvents and chemicals are used for syntheses.

HPLC Condition

Column: Nucleosil 100-3 C18 HD, 125×4.0 mm.

Flow rate: 1.0 ml/min

Mobile phase: A) TFA/water (0.1/100, v/v), B) TFA/acetonitrile (0.1/100, v/v)

Gradient: linear gradient from 20% B to 100% B in 7 min

Detection: UV at 254 nm

The HPLC conditions can be identified by the subscript prefixes of the T_{Ret} values given in the examples.

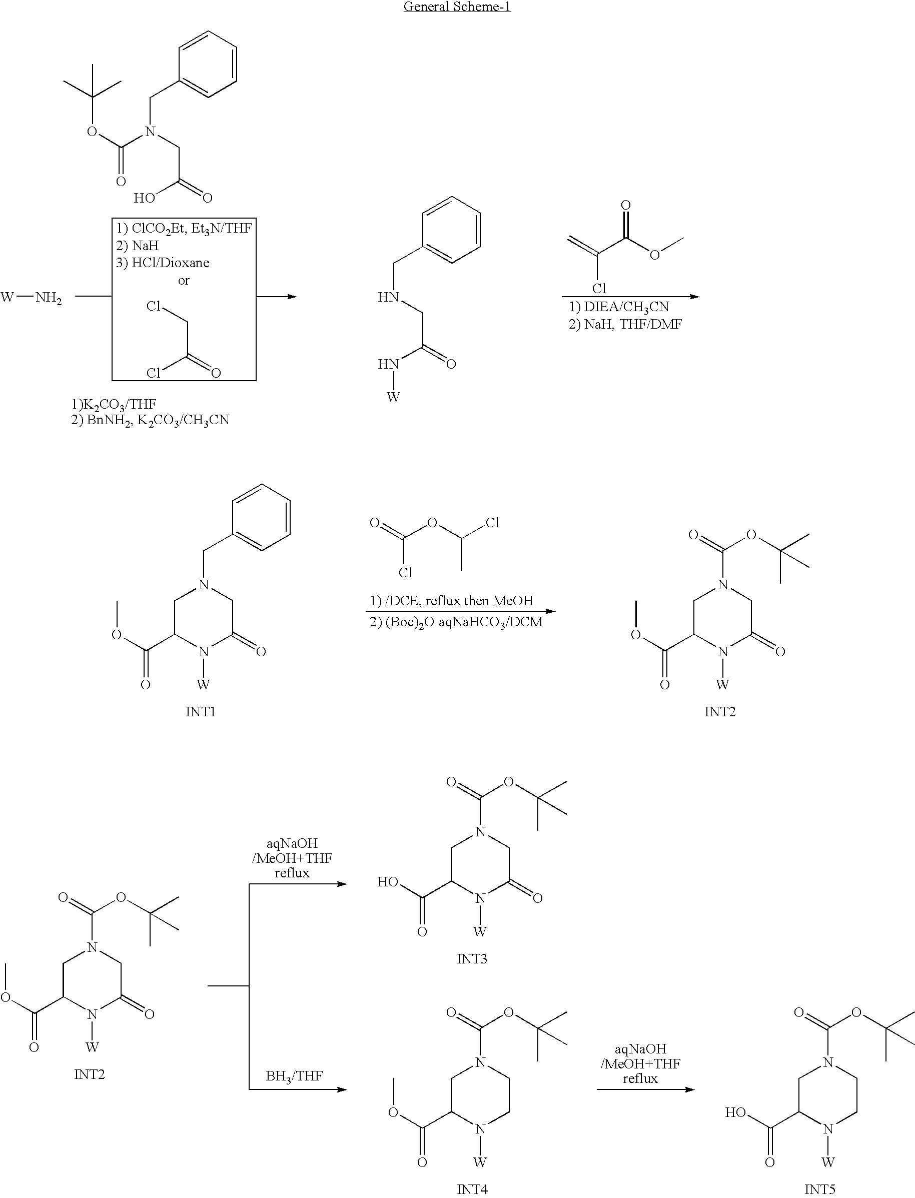

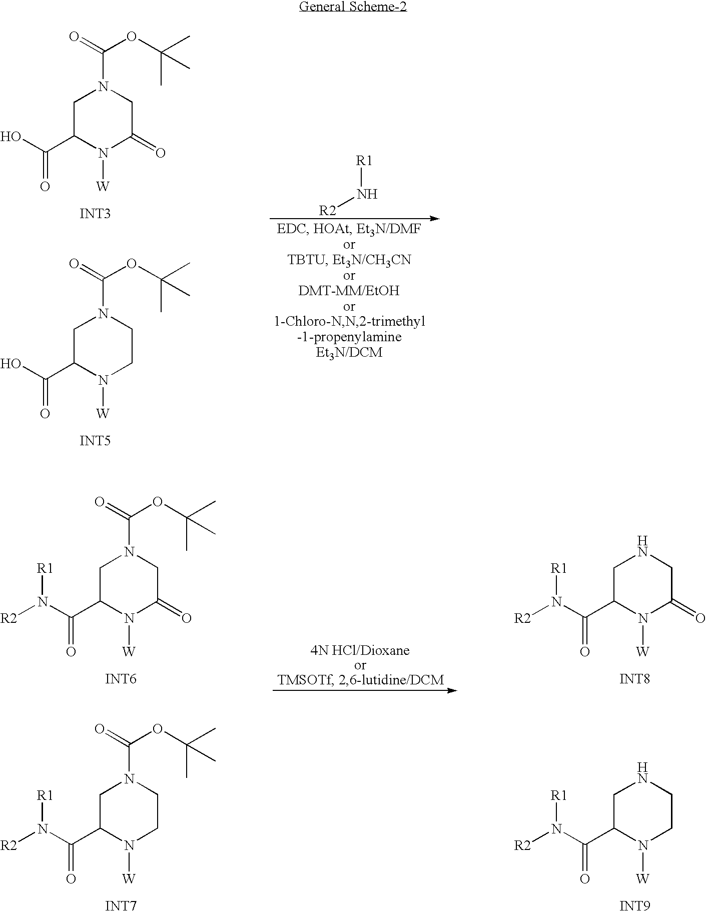

Intermediates INT1, INT2, INT3, INT4, INT5, INT6, INT7, INT8, INT9 are obtained as a racemic mixture, or optical resolution of INT1 using an appropriate chiral acid (such as tartaric acid) or INT3 and INT5 using an appropriate chiral amine (such as cinchonidine, cinchonine, quinine or quinidine) affords corresponding enantiomeric pure INT1 or INT3 or INT5. Alternatively or in addition, the final product INT8 or INT9 can be separated into the pure enantiomers by common techniques like chiral chromatography.

### EXAMPLE 1

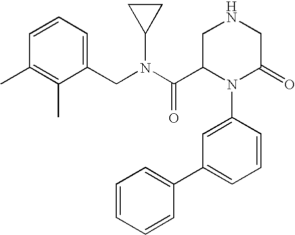

A mixture of Intermediate 1.1 (159 mg, 0.28 mmol) and 4N dioxane solution of HCl (3 mL) is stirred under N₂ at RT. After stirring for 30 min, the reaction mixture is concentrated under reduced pressure to give Example 1 as white solid; ES-MS: M+H=454; HPLC: t_{Ret}=3.48 min

Intermediate 1.1:

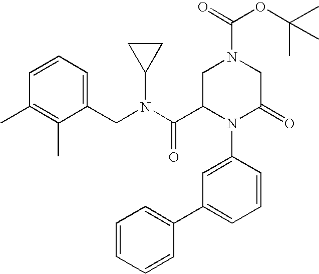

A mixture of Intermediate 1.2 (110 mg, 0.29 mmol), cyclopropyl(2,3-dimethylbenzyl)amine and TBTU (160 mg, 0.42 mol) in CH₃CN (3 mL), Et₃N (0.16 mL, 1.2 mmol) is stirred for 1 h at room temperature. EtOAc is added and the organic layer is washed with brine, dried over MgSO₄ and evaporated in vacuo. Silica gel flash chromatography of the residue affords Intermediate 1.1 as an oil; ES-MS: M+H=554; HPLC: t_{Ret}=4.80 min.

Intermediate 1.2:

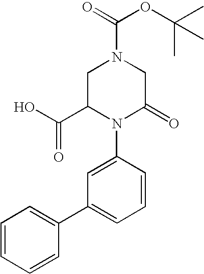

A mixture of Intermediate 1.3 (300 mg, 0.73 mmol) in MeOH (4 mL) and aqueous 2M NaOH (2 mL) is refluxed for 30 min. After aqueous 1M HCl, the reaction mixture is extracted with EtOAc. The combined organic phases are washed with H₂O, brine and dried (Na₂SO₄). Silica gel flash chromatography of the residue affords Intermediate 1.2 as an oil; ES-MS: M+H=397; HPLC: t_{Ret}=3.46 min.

Intermediate 1.3:

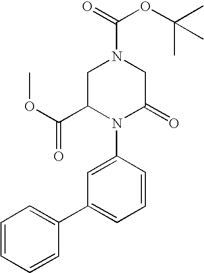

A mixture of Intermediate 1.4 (50 mg, 0.16 mmol) and (Boc)₂O (44 μL, 0.19 mmol) in DCM (2 mL) and aqueous saturated NaHCO₃ (0.5 mL) is stirred for 2 h at room temperature. After adding H₂O, the reaction mixture is extracted with DCM. The combined organic phases are washed with H₂O, brine and dried (Na₂SO₄). Silica gel flash chromatography of the residue affords Intermediate 1.3 as an oil; ES-MS: M+H=411; HPLC: t_{Ret}=4.02 min.

Intermediate 1.4:

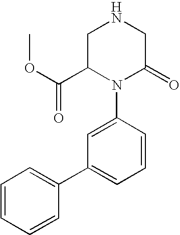

A mixture of Intermediate 1.5 (70 mg, 0.17 mmol) and chloroformic acid-1-chloro methylester (80 μL, 0.70 mmol) in 1,2-dichloroethane (2 mL) is stirred for 6 h at 90° C. After evaporation in vacuo, MeOH (4 mL) is added and the reaction mixture is refluxed for 2 h. After evaporation, silica gel flash chromatography of the residue affords Intermediate 1.4 as an oil; ES-MS: M+H=311; HPLC: t_{Ret}=2.64 min.

Intermediate 1.5:

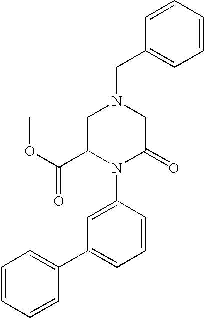

A mixture of Intermediate 1.6 (1.80 g, 4.12 mmol) and NaH (200 mg, 4.94 mmol) in THF (15 mL) and DMF (5 mL) is stirred for 30 min at room temperature and 10 min at 50° C. After adding H₂O, the reaction mixture is extracted with EtOAc. The combined organic phases are washed with H₂O, brine and dried (Na₂SO₄). Silica gel flash chromatography of the residue affords Intermediate 1.5 as an oil; ES-MS: M+H=401; HPLC: t_{Ret}=4.02 min.

Intermediate 1.6:

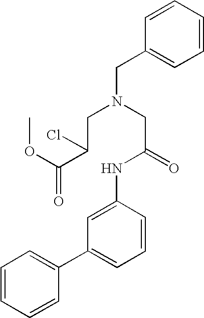

A mixture of Intermediate 1.7 (300 mg, 0.85 mmol), methyl-α-chloroacrylate (0.1 mL, 1.28 mmol) and DIEA (0.3 mL, 1.67 mol) in CH₃CN (3 mL) is stirred for 6 h at 60° C. After evaporation in vacuo, silica gel flash chromatography of the residue affords Intermediate 1.6 as an oil; ES-MS: M+H=437; HPLC: t_{Ret}=4.54 min.

Intermediate 1.7:

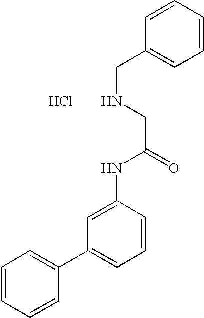

A mixture of N-benzyl-N-tert-butoxycarbonylglycine (1.0 g, 3.77 mmol) (see e.g. J. Am. Chem. Soc. 125, 10664, 2003) and chloroformic acid ethylester (0.4 mL, 4.14 mmol) in THF (20 mL), Et₃N (0.63 mL, 4.52 mmol) is stirred at room temperature. After stirring for 30 min, the reaction mixture is filtered for removing inorganic salt. A solution of this crude product in THF is treated with 3-aminobiphenyl (765 mg, 4.52 mmol) and NaH (230 mg, 0.5.7 mmol) at room temperature for 30 min. After adding H₂O, the reaction mixture is extracted with EtOAc. The combined organic phases are washed with H₂O, brine and dried (Na₂SO₄). Concentration under reduced pressure affords a crude product. Then deprotection of this crude product by 4M dioxane solution of HCl (20 mL) at room temperature affords Intermediate 1.7 as white solid; ES-MS: M+H=317; HPLC: t_{Ret}=3.09 min.

### EXAMPLE 2

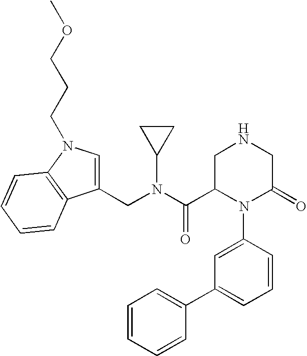

Example 2 is synthesized by deprotection of Intermediate 2.1 (247 mg, 0.39 mmol) analogously to the preparation of Example 1. White solid; ES-MS: M+H=537; HPLC: t_{Ret}=3.46 min.

Intermediate 2.1:

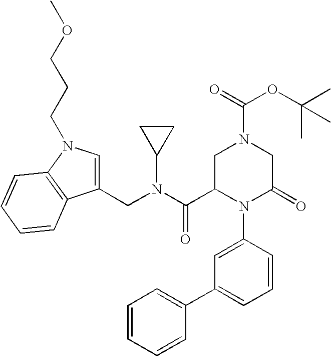

Intermediate 2.1 is synthesized by condensation of Intermediate 1.2 (150 mg, 0.39 mmol) and Intermediate 2.2 (131 mg, 0.50 mmol) analogously to the preparation of Example 1. White solid; ES-MS: M+H=637; HPLC: t_{Ret}=4.67 min.

Intermediate 2.2:

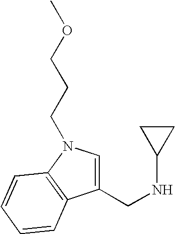

A mixture of Intermediate 2.3 (780 mg, 3.6 mmol), cyclopropylamine (410 mg, 7.2 mmol), AcOH (0.5 mL) and NaBH(OAc)₃ (1.1 g, 5.4 mmol) in DCM (3 mL) and MeOH (1 mL) is stirred under N₂ at 0° C. After stirring at RT for 1 h, the reaction mixture is quenched with saturated aqueous NaHCO₃ and extracted with DCM. The combined organic phases are washed with H₂O, brine and dried (Na₂SO₄). Concentration under reduced pressure and silica gel flash chromatography give Intermediate 2.2 as yellow oil; ES-MS: M+H=202; HPLC: t_{Ret}=2.67 min

Intermediate 2.3:

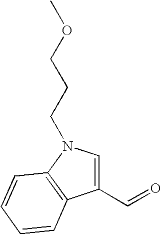

To a mixture of indole-3-carboxaldehyde (1.0 g, 6.9 mmol), toluene-4-sulfonic acid 3-methoxy-propyl ester (2.1 g, 9.0 mmol) and KI (1.1 g, 7.0 mmol) in DMF (15 mL), NaH (320 mg, 7.5 mmol) is added under N₂ at 0° C. After stirring at 50° C. for 4 h, the H₂O is added to the reaction mixture which is then extracted with EtOAc. The combined organic phases are washed with H₂O, brine and dried (Na₂SO₄). Concentration under reduced pressure and silica gel flash chromatography give Intermediate 2.3 as colorless oil; ES-MS: M+H=218, HPLC: t_{Ret}=3.18 min.

The following Examples enlisted on Table 1 are synthesized analogously to the preparation of Example 1-2. As far as not being commercially available, the synthesis of intermediates for the preparation of compounds of Examples 3-15 is described below the Table 1. The asterisk (*) marks the end of the bond via at which a moiety is bound to the rest of the molecule:

TABLE 1

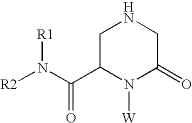

| Example | R1 | R2 | W | Analytical data |
|---|---|---|---|---|
| 3 | 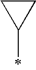 | 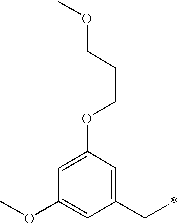 | 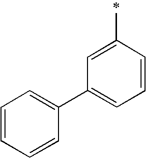 | MS: [M + 1]⁺ = 544HPLC t_{Ret} = 3.40 min. |
| 4 | 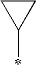 | 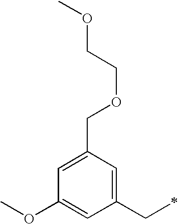 | 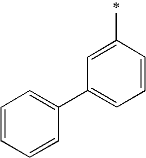 | MS: [M + 1]⁺ = 544HPLC t_{Ret} = 3.13 min. |
| 5 | 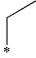 | 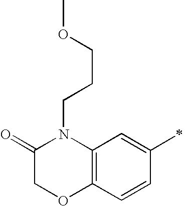 | 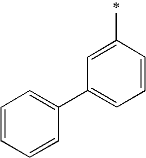 | MS: [M + 1]⁺ = 543HPLC t_{Ret} = 3.09 min. |
| 6 | 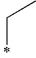 | 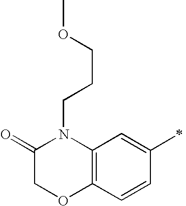 | 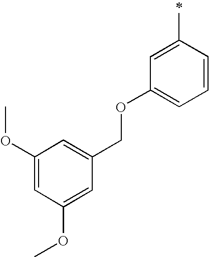 | MS: [M + 1]⁺ = 633HPLC t_{Ret} = 3.36 min. |
| 7 | 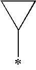 | 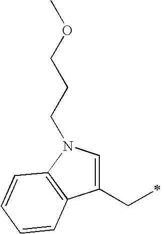 | 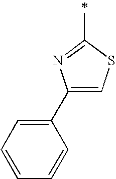 | MS: [M + 1]⁺ = 544HPLC t_{Ret} = 3.67 min. |
| 8 | 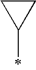 | 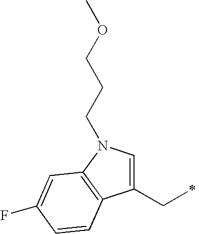 | 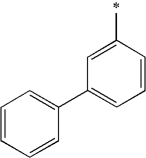 | MS: [M + 1]⁺ = 555HPLC t_{Ret} = 3.57 min. |
| 9 | 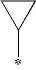 | 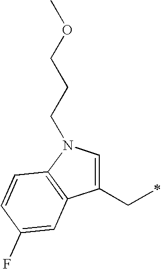 | 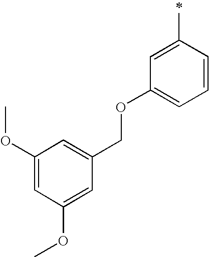 | MS: [M + 1]⁺ = 645HPLC t_{Ret} = 3.79 min. |
| 10 | 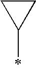 | 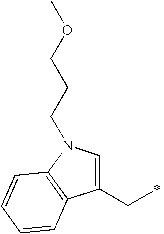 | 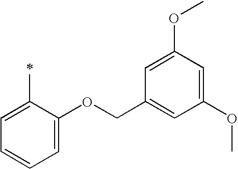 | MS: [M + 1]⁺ = 627HPLC t_{Ret} = 3.48 min. |
| 11 | 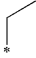 | 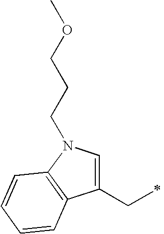 | 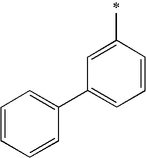 | MS: [M + 1]⁺ = 525HPLC t_{Ret} = 3.32 min. |
| 12 | H | 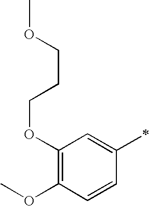 | 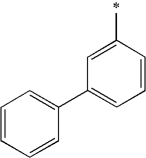 | MS: [M + 1]⁺ = 490HPLC t_{Ret} = 2.69 min. |
| 13 | 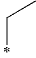 | 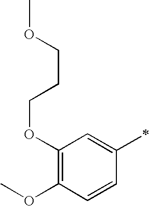 | 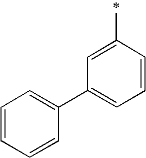 | MS: [M + 1]⁺ = 518HPLC t_{Ret} = 2.89 min. |
| 14 | H | 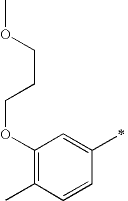 | 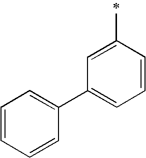 | MS: [M + 1]⁺ = 474HPLC t_{Ret} = 3.26 min. |
| 15 | 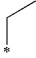 | 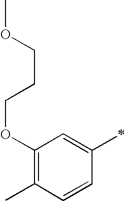 | 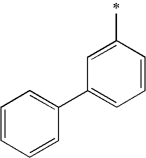 | MS: [M + 1]⁺ = 502HPLC t_{Ret} = 3.42 min. |

Intermediate 3.1:

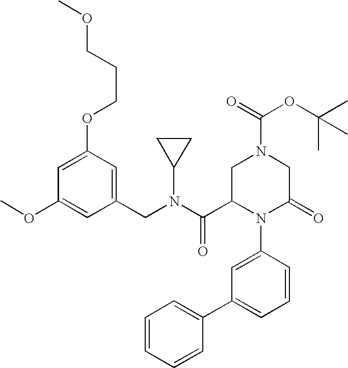

Intermediate 3.1 is synthesized by condensation of Intermediate 1.2 (205 mg, 0.52 mmol) and Intermediate 3.2 (178 mg, 0.67 mmol) analogously to the preparation of Intermediate 1.1. White solid; ES-MS: M-^{t}Bu=588; HPLC: t_{Ret}=4.67 min.

Intermediate 3.2:

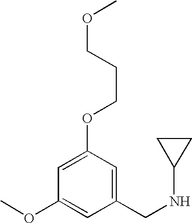

Intermediate 3.2 is synthesized by condensation of Intermediate 3.3 (2.50 g, 11.1 mmol) and cyclopropylamine (1.16 mL, 16.7 mmol) analogously to the preparation of Intermediate 2.2. Yellow oil; ES-MS: M+H=266; HPLC: t_{Ret}=2.48 min.

Intermediate 3.3:

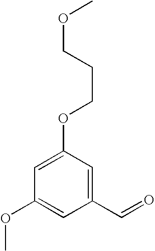

A mixture of Intermediate 3.4 (4.2 g, 18.6 mmol) and MnO₂ (15 g, excess) in toluene (100 mL) is stirred under N₂ at room temperature for overnight. After filtration removing MnO₂, the filtrate is concentrated under reduced pressure and silica gel flash chromatography to give Intermediate 3.3 as colorless oil; ES-MS: M+H=225; HPLC: t_{Ret}=3.59 min.

Intermediate 3.4:

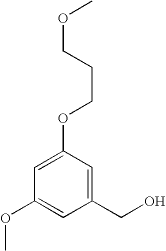

A mixture of Intermediate 3.5 (5 g, 19.7 mmol) and LAH (528 mg, 20 mmol) in THF (110 mL) is stirred under N₂ at 0° C. for 3 h. After adding H₂O, the reaction mixture is extracted with EtOAc. The combined organic phases are washed with H₂O, brine and dried (Na₂SO₄). Concentration under reduced pressure and silica gel flash chromatography give Intermediate 3.4 as colorless oil; ES-MS: M+H=227; HPLC: t_{Ret}=2.85 min.

Intermediate 3.5:

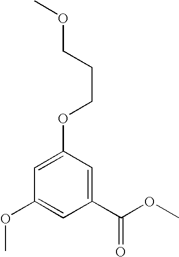

To a mixture of 3-methoxy-5-hydroxybenzoic acid methyl ester (23.2 g, 127 mmol toluene-4-sulfonic acid 3-methoxy-propyl ester (40.7 g, 167 mmol) and KI (2.23 g, 13.4 mmol) in DMF (350 mL), K₂CO₃ (53.1 g, 384 mmol) is added under N₂. After stirring at 60° C. for 17 h, the reaction mixture is supplemented with H₂O and extracted with Et₂O. The combined organic phases are washed with H₂O and dried (Na₂SO₄). Concentration under reduced pressure and silica gel flash chromatography give Intermediate 3.5 as colorless oil; ES-MS: M+H=255, HPLC: t_{Ret}=3.80 min.

Intermediate 4.1:

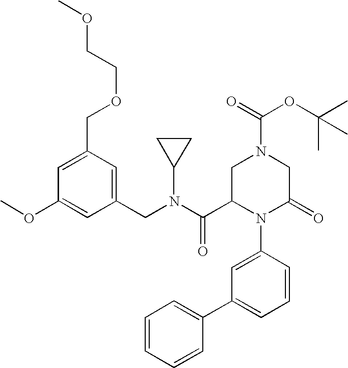

Intermediate 4.1 is synthesized by condensation of Intermediate 1.2 (210 mg, 0.53 mmol) and Intermediate 4.2 (144 mg, 0.69 mmol) analogously to the preparation of Intermediate 1.1. White solid; ES-MS: M-^{t}Bu=588; HPLC: t_{Ret}=4.40 min.

Intermediate 4.2:

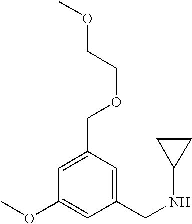

Intermediate 4.2 is synthesized by condensation of Intermediate 4.3 (10.3 g, 45.9 mmol) and cyclopropylamine (6.4 mL, 91.8 mmol) analogously to the preparation of Intermediate 2.2. Colorless oil; Rf=0.20 (AcOEt:DCM=2:1); ¹H NMR (CDCl₃) δ 0.33-0.45 (m, 4H), 2.12-2.18 (m, 1H), 3.39 (s, 3H), 3.54-3.63 (m, 4H), 3.79 (s, 3H), 4.54 (s, 2H), 6.75 (s, 1H), 6.77 (s, 1H), 6.85 (s, 1H).

Intermediate 4.3:

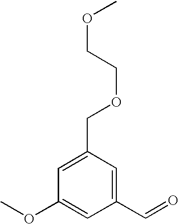

A mixture of Intermediate 4.4 (12.9 g, 57 mmol) and MnO₂ (17.5 g, excess) in EtOAc (200 mL) is stirred under N₂ at 60° C. for 4 h. After filtration removing MnO₂, the filtrate is concentrated under reduced pressure and silica gel flash chromatography to give Intermediate 4.3 as colorless oil; Rf=0.45 (AcOEt:n-Hex=1:1); ¹H NMR (CDCl₃) δ 3.39 (s, 3H), 3.56-3.68 (m, 4H), 3.87 (s, 3H), 4.61 (s, 2H), 7.19 (s, 1H), 7.30 (s, 1), 7.47 (s, 1H), 9.98 (s, 1H).

Intermediate 4.4:

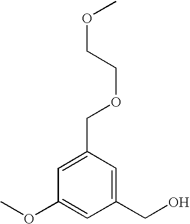

Intermediate 4.4 is synthesized by reduction of Intermediate 4.5 (824 mg, 3.3 mmol) analogously to the preparation of Intermediate 3.4. White powder; HPLC: t_{Ret}=2.52 min; Rf=0.21 (EtOAc:n-Hex=1:1)

Intermediate 4.5:

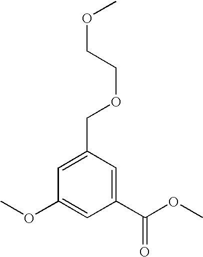

Intermediate 4.5 is synthesized by alkylation of 3-(hydroxymethyl)-5-methoxy-benzoic acid methylester (1.85 g, 9.4 mmol) (see e.g. Synthetic Communications, 2001, 31, 1921-1926) analogously to the preparation of Intermediate 2.3. White amorphous material; ES-MS: M+H=255; HPLC: t_{Ret}=3.44 min

Intermediate 5.1:

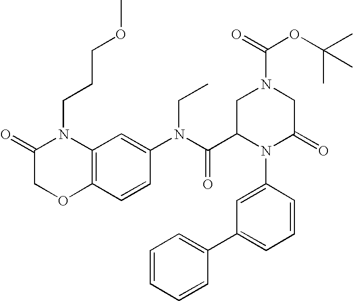

A mixture of Intermediate 1.2 (119 mg, 0.30 mmol) and 1-chloro-N,N,2-trimethyl-1-propenylamine (60 μl, 0.45 mmol) in DCM (3 mL) is stirred for 1 h at room temperature. After concentration in vacuo, a mixture of Et₃N (0.1 mL, 0.72 mmol) and Intermediate 5.2 (99 mg, 0.33) in THF (3 mL) is stirred at 60° C. for overnight. After adding H₂O, the reaction mixture is extracted with EtOAc. The combined organic phases are washed with H₂O, brine and dried (Na₂SO₄). Concentration under reduced pressure and silica gel flash chromatography give Intermediate 5.1 as colorless oil; ES-MS: M+H=643; HPLC: t_{Ret}=4.32 min.

Intermediate 5.2:

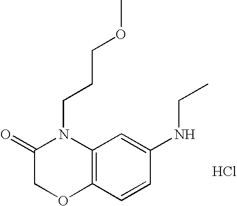

Intermediate 5.3 (1.08 g, 2.96 mmol) is treated with 4M dioxane solution of HCl (10 mL) at room temperature. After stirring for 2 h, concentration under reduced pressure give Intermediate 5.2 as white powder; ES-MS: M+H=265; HPLC: t_{Ret}=2.05 min.

Intermediate 5.3:

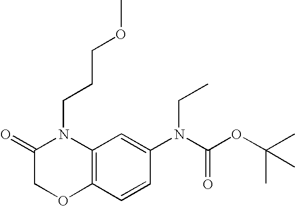

A mixture of Intermediate 5.4 (1.59 g, 4.72 mmol), EtI (0.41 mL, 5.19 mmol) and NaH (208 mg, 5.19 mmol) in THF (20 mL) is stirred at room temperature for overnight. After adding H₂O, the reaction mixture is extracted with EtOAc. The combined organic phases are washed with H₂O, brine and dried (Na₂SO₄). Concentration under reduced pressure and silica gel flash chromatography give Intermediate 5.3 as colorless oil; ES-MS: M+H-^{t}Bu=309; HPLC: t_{Ret}=4.03 min.

Intermediate 5.4:

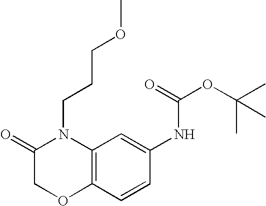

A mixture of Intermediate 5.5 (1.36 g, 5.74 mmol) and (Boc)₂O (1.56 g, 28 mmol) in THF (20 mL) and Et₃N (0.96 mL, 6.88 mmol) is stirred at room temperature. After stirring for 2 h. After adding H₂O, the reaction mixture is extracted with EtOAc. The combined organic phases are washed with H₂O, brine and dried (Na₂SO₄). Concentration under reduced pressure and silica gel flash chromatography give Intermediate 5.4 as colorless solid; ES-MS: M+H=337; HPLC: t_{Ret}=3.67 min.

Intermediate 5.5:

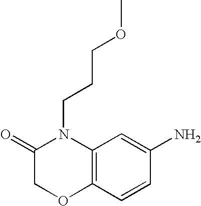

A mixture of Intermediate 5.6 (2.48 g, 9.33 mmol) and Fe powder (1.56 g, 28 mmol) in EtOH (30 mL) and 5M aqueous solution of HCl (5.6 mL) is stirred at 70° C. After stirring for overnight, the reaction mixture is filtered by Celite. After adding H₂O, the reaction mixture is extracted with EtOAc. The combined organic phases are washed with H₂O, brine and dried (Na₂SO₄). Concentration under reduced pressure and silica gel flash chromatography give Intermediate 5.5 as brown solid; ES-MS: M+H=237; HPLC: t_{Ret}=1.82 min.

Intermediate 5.6:

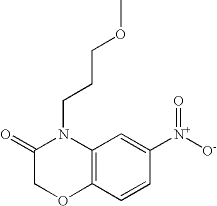

Intermediate 5.6 is synthesized by alkylation of 6-nitro-2H-1,4-benzoxazin-3(4H)-one (582 mg, 3.00 mmol) and toluene-4-sulfonic acid 3-methoxy-propyl ester (1.1 g, 4.50 mmol) analogously to the preparation of Intermediate 2.3. Brown oil; ES-MS: M+H=267; HPLC: t_{Ret}=3.18 min.

Intermediate 6.1:

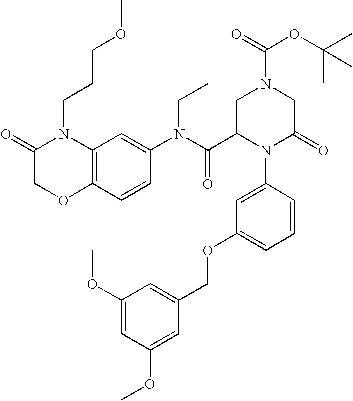

Intermediate 6.1 is synthesized by condensation of Intermediate 6.2 (200 mg, 0.41 mmol) and Intermediate 5.2 (141 mg, 0.53 mmol) analogously to the preparation of Intermediate 5.1. White solid; ES-MS: M+H=733; HPLC: t_{Ret}=4.47 min.

Intermediate 6.2:

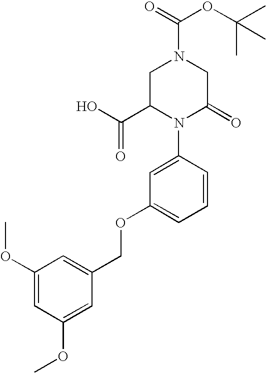

Intermediate 6.2 is synthesized by hydrolysis of Intermediate 6.3 (767 mg, 1.53 mmol) analogously to the preparation of Intermediate 1.2. White solid; ES-MS: M+H=487; HPLC: t_{Ret}=3.77 min.

Intermediate 6.3:

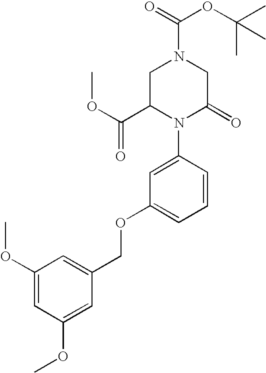

Intermediate 6.4 is synthesized by alkylation of Intermediate 6.5 (600 mg, 1.71 mmol) and 3,5-dimethoxybenzyl bromide (594 mg, 2.56 mmol) analogously to the preparation of Intermediate 2.3. White amorphous material; ES-MS: M+H=501; HPLC: t_{Ret}=4.28 min.

Intermediate 6.4:

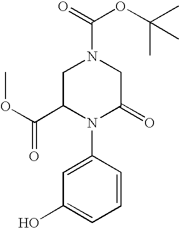

Intermediate 6.4 is synthesized by deprotection and protection of Intermediate 6.5 (8.0 g, 20.8 mmol) analogously to the preparation of Intermediate 1.4 and 1.3. White solid; ES-MS: M+H=351; HPLC: t_{Ret}=3.29 min.

Intermediate 6.5:

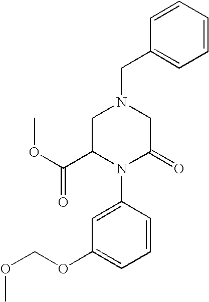

Intermediate 6.5 is synthesized by cyclization of Intermediate 6.6 (13.6 g, 32.3 mmol) analogously to the preparation of Intermediate 1.5. White solid; ES-MS: M+H=385; HPLC: t_{Ret}=3.63 min.

Intermediate 6.6:

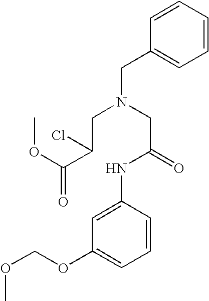

Intermediate 6.6 is synthesized by 1,4-addition of Intermediate 6.7 (20.0 g, 66.6 mmol) analogously to the preparation of Intermediate 1.6. White solid; ES-MS: M+H=421; HPLC: t_{Ret}=4.14 min.

Intermediate 6.7:

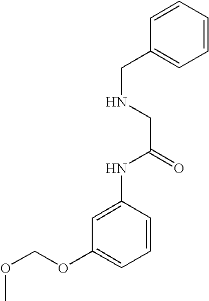

A mixture of Intermediate 6.8 (25 g, 108 mmol), benzylamine (23.4 g, 280 mmol), K₂CO₃ (32.8 g, 240 mmol) and KI (1.38 g, 10 mmol) in CH₃CN (540 mL) is stirred at room temperature for overnight. After concentration under reduced pressure, the reaction mixture is extracted with Et₂O. The combined organic phases are washed with H₂O, brine and dried (Na₂SO₄). Concentration under reduced pressure and silica gel flash chromatography give Intermediate 6.7 as colorless oil; ES-MS: M+H=301; HPLC: t_{Ret}=2.75 min.

Intermediate 6.8:

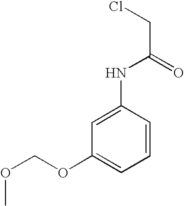

A mixture of 2-chloro-3′-hydroxyacetanilide (25 g, 134.6 mmol) (see Egyptian Journal of Pharmaceutical Sciences 1991, 32, 251-61), MOMCl (20.9 mL, 269.4 mmol) and DIEA (71.0 mL, 539 mmol) in DCM (270 mL) is stirred at room temperature for 1 h. After adding H₂O, the reaction mixture is extracted with DCM. The combined organic phases are washed with H₂O, brine and dried (Na₂SO₄). Concentration under reduced pressure and silica gel flash chromatography give Intermediate 6.8 as colorless oil; ES-MS: M+H=230; HPLC: t_{Ret}=3.09 min.

Intermediate 7.1:

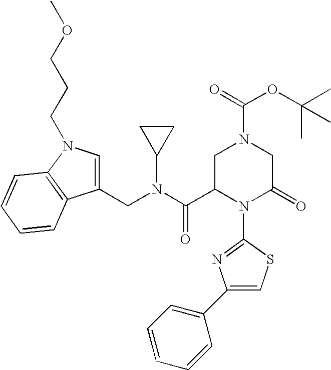

Intermediate 7.1 is synthesized by condensation of Intermediate 7.2 (146 mg, 0.36 mmol) and Intermediate 2.2 (121 mg, 0.47 mmol) analogously to the preparation of Intermediate 1.1. White solid; ES-MS: M+H=644; HPLC: t_{Ret}=5.25 min.

Intermediate 7.2:

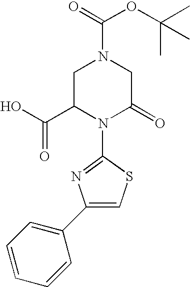

Intermediate 7.2 is synthesized by hydrolysis of Intermediate 7.3 (190 mg, 0.46 mmol) analogously to the preparation of Intermediate 1.2. White amorphous material; ES-MS: M+H+H₂O=422; HPLC: t_{Ret}=3.02 min.

Intermediate 7.3:

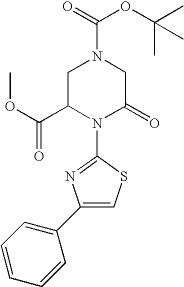

Intermediate 7.3 is synthesized by deprotection and protection of Intermediate 7.4 (620 mg, 1.52 mmol) analogously to the preparation of Intermediate 1.4 and 1.3. White solid; ES-MS: M+H=318; HPLC: t_{Ret}=2.83 min.

Intermediate 7.4:

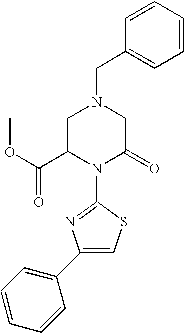

Intermediate 7.4 is synthesized by 1,4-addition and cyclization of Intermediate 7.5 (1.48 g, 4.1 mmol) analogously to the preparation of Intermediate 1.6 and 1.5. White solid; ES-MS: M+H=408; HPLC: t_{Ret}=4.81 min.

Intermediate 7.5:

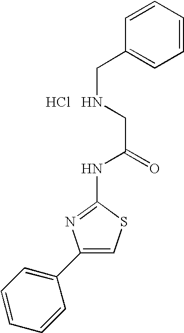

Intermediate 7.5 is synthesized by condensation and deprotection of 2-amino-4-phenyl thiazole (1.95 g, 10.7 mmol) and N-benzyl-N-tert-butoxycarbonylglycine (3.0 g, 11.3 mmol) analogously to the preparation of Intermediate 1.7. White solid; ES-MS: M+H=324; HPLC: t_{Ret}=2.93 min.

Intermediate 8.1:

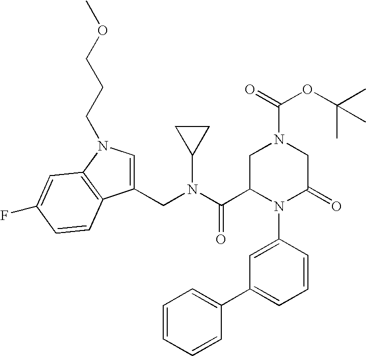

Intermediate 8.1 is synthesized by condensation of Intermediate 6.2 (202 mg, 0.51 mmol) and Intermediate 8.2 (183 mg, 0.66 mmol) analogously to the preparation of Intermediate 1.1. White solid; ES-MS: M+H=655; HPLC: t_{Ret}=4.84 min.

Intermediate:

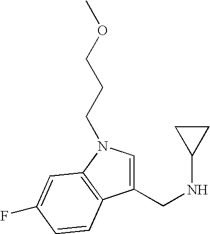

Intermediate 8.2 is synthesized by condensation of Intermediate 8.3 (1.20 g, 5.10 mmol) and cyclopropylamine (581 mg, 10.2 mmol) analogously to the preparation of Intermediate 2.2. Yellow oil; ES-MS: M−H=275; HPLC: t_{Ret}=2.57 min.

Intermediate 8.3:

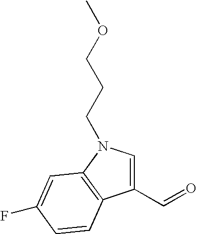

Intermediate 8.3 is synthesized by condensation of 6-fluoro-indole-3-carbaldehyde (1.00 g, 7.40 mmol) and toluene-4-sulfonic acid 3-methoxy-propyl ester (2.30 g, 9.60 mmol) analogously to the preparation of Intermediate 2.3. Yellow oil; ES-MS: M+H=236; HPLC: t_{Ret}=3.27 min.

Intermediate 9.1:

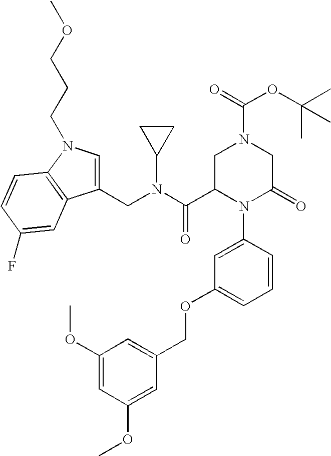

Intermediate 9.1 is synthesized by condensation of Intermediate 6.2 (200 mg, 0.31 mmol) and Intermediate 9.2 (147 mg, 0.53 mmol) analogously to the preparation of Intermediate 1.1. White solid; ES-MS: M+H=745; HPLC: t_{Ret}=5.08 min.

Intermediate 9.2:

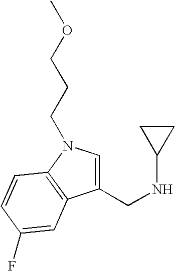

Intermediate 9.2 is synthesized by condensation of Intermediate 9.3 (640 mg, 2.70 mmol) and cyclopropylamine (308 mg, 5.40 mmol) analogously to the preparation of Intermediate 2.2. Colorless oil; ES-MS: M+H=277; HPLC: t_{Ret}=2.57 min.

Intermediate 9.3:

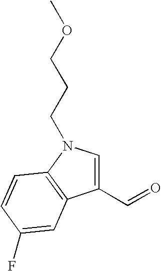

Intermediate 9.3 is synthesized by condensation of 5-fluoro-indole-3-carbaldehyde (500 mg, 3.10 mmol) and toluene-4-sulfonic acid 3-methoxy-propyl ester (973 mg, 3.90 mmol) analogously to the preparation of Intermediate 2.3. Yellow oil; ES-MS: M+H=236; HPLC: t_{Ret}=3.22 min.

Intermediate 10.1:

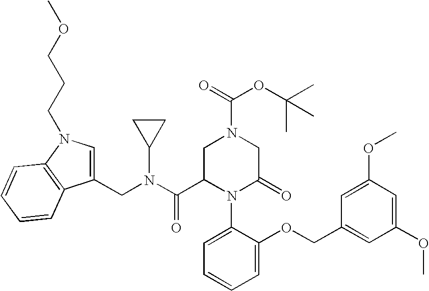

Intermediate 10.1 is synthesized by condensation of Intermediate 10.2 (193 mg, 0.40 mmol) and Intermediate 2.2 (134 mg, 0.52 mmol) analogously to the preparation of Intermediate 1.1. White amorphous material; ES-MS: M+H=727; HPLC: t_{Ret}=4.87 min.

Intermediate 10.2:

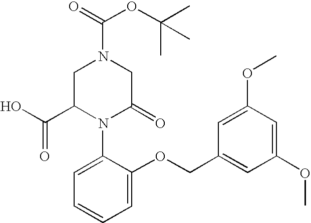

Intermediate 10.2 is synthesized by hydrolysis of Intermediate 10.3 (200 mg, 0.40 mmol) analogously to the preparation of Intermediate 1.2. White amorphous material; ES-MS: M+H=487; HPLC: t_{Ret}=3.66 min.

Intermediate 10.3:

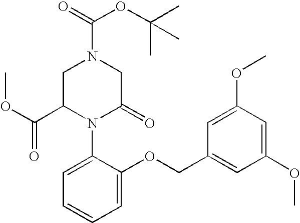

Intermediate 10.3 is synthesized by alkylation of Intermediate 10.4 (400 mg, 1.14 mmol) and 3,5-dimethoxybenzyl bromide (393 mg, 1.70 mmol) analogously to the preparation of Intermediate 2.3. White amorphous material; ES-MS: M+H=501; HPLC: t_{Ret}=4.18 min.

Intermediate 10.4:

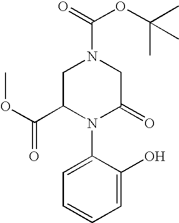

Intermediate 10.4 is synthesized by deprotection and protection of Intermediate 10.5 (500 mg, 1.30 mmol) analogously to the preparation of Intermediate 1.4 and 1.3. White solid; ES-MS: M+H=351; HPLC: t_{Ret}=3.12 min.

Intermediate 10.5:

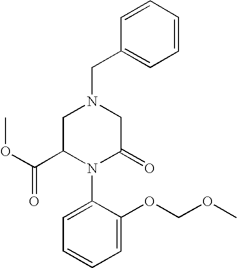

Intermediate 10.5 is synthesized by cyclization of Intermediate 10.6 (7.15 g, 17.0 mmol) analogously to the preparation of Intermediate 1.5. White solid; ES-MS: M+H=385; HPLC: t_{Ret}=3.42 min.

Intermediate 10.6:

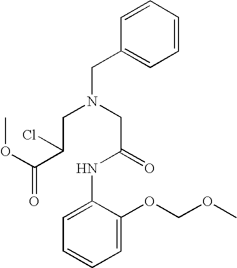

Intermediate 10.6 is synthesized by 1,4-addition of Intermediate 10.7 (9.21 g, 30.7 mmol) analogously to the preparation of Intermediate 1.6. White amorphous material; ES-MS: M+H=421; HPLC: t_{Ret}=4.24 min.

Intermediate 10.7:

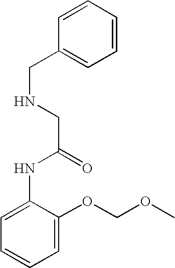

Intermediate 10.7 is synthesized by amination of Intermediate 10.8 (7.45 g, 32.4 mmol) analogously to the preparation of Intermediate 6.7. White amorphous material; ES-MS: M+H=301; HPLC: t_{Ret}=2.40 min.

Intermediate 10.8:

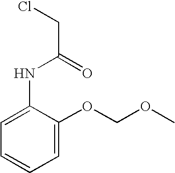

A mixture of 2-chloro-2′-hydroxyacetanilide (8.0 g, 43.1 mmol) (see Egyptian Journal of Pharmaceutical Sciences 1991, 32, 251-61), MOMCl (5.1 mL, 65 mmol) and DIEA (22.5 mL, 129 mmol) in DCM (86 mL) is stirred at room temperature for 1h. After adding H₂O, the reaction mixture is extracted with DCM. The combined organic phases are washed with H₂O, brine and dried (Na₂SO₄). Concentration under reduced pressure and silica gel flash chromatography give Intermediate 10.8 as colorless oil; ES-MS: M+H=230; HPLC: t_{Ret}=3.15 min.

Intermediate 11.1:

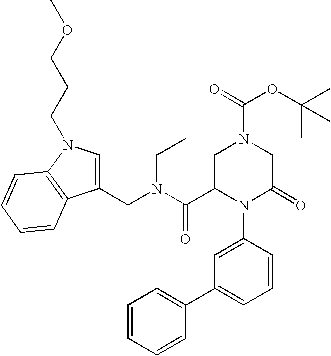

Intermediate 11.1 is synthesized by condensation of Intermediate 1.2 (25 mg, 0.063 mmol) and Intermediate 11.2 (20.2 mg, 0.095 mmol) analogously to the preparation of Intermediate 1.1. White amorphous material; ES-MS: M+H=625; HPLC: t_{Ret}=4.59 min.

Intermediate 11.2:

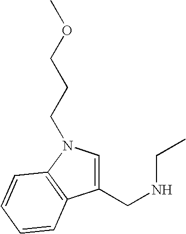

Intermediate 11.2 is synthesized by reductive amination of Intermediate 2.3 (3.0 g, 13.8 mmol) and 2M THF solution of ethylamine (10.3 mL, 20.6 mmol) analogously to the preparation of Intermediate 2.2. White amorphous material; ES-MS: M+H=246; HPLC: t_{Ret}=2.36 min.

Intermediate 12.1:

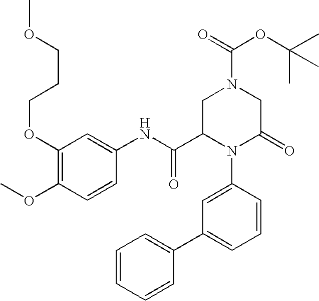

Intermediate 12.1 is synthesized by condensation of Intermediate 1.2 (300 mg, 0.76 mmol) and Intermediate 12.2 (244 mg, 0.98 mmol) analogously to the preparation of Intermediate 1.1. White amorphous material; ES-MS: M-^{t}Boc=534; HPLC: t_{Ret}=3.70 min.

Intermediate 12.2:

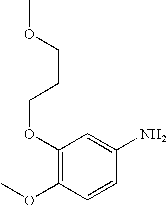

Intermediate 12.2 is synthesized by reduction of Intermediate 12.3 (1.6 g, 6.63 mmol) analogously to the preparation of Intermediate 5.5. White amorphous material; ES-MS: M+H=212; HPLC: t_{Ret}=2.19 min.

Intermediate 12.3:

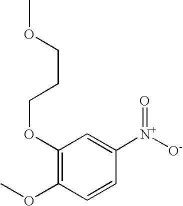

Intermediate 12.3 is synthesized by alkylation of 5-nitroguaiacol (5.0 g, 29.6 mmol) analogously to the preparation of Intermediate 3.5. White amorphous material; ES-MS: M+H=242; HPLC: t_{Ret}=3.63 min.

Intermediate 13.1:

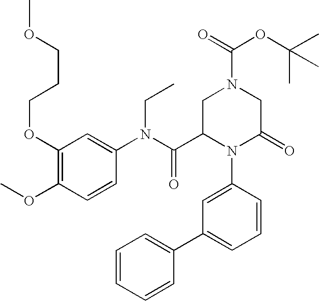

Intermediate 13.1 is synthesized by alkylation of Intermediate 12.1 (230 mg, 0.39 mmol) analogously to the preparation of Intermediate 5.3. White amorphous material; ES-MS: M+H=618; HPLC: t_{Ret}=4.03 min.

Intermediate 14.1:

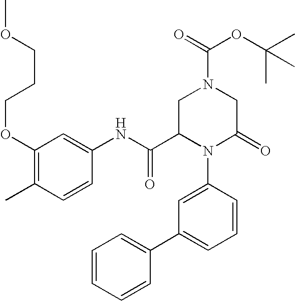

Intermediate 14.1 is synthesized by condensation of Intermediate 1.2 (300 mg, 0.76 mmol) and Intermediate 14.2 (194 mg, 0.99 mmol) analogously to the preparation of Intermediate 1.1. White amorphous material; ES-MS: M-^{t}Boc=318; HPLC: t_{Ret}=4.45 min.

Intermediate 14.2:

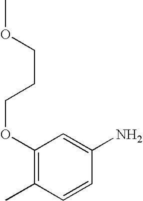

Intermediate 14.2 is synthesized by reduction of Intermediate 14.3 (8.4 g, 37.3 mmol) analogously to the preparation of Intermediate 5.5. White amorphous material; ES-MS: M+H=196; HPLC: t_{Ret}=2.19 min.

Intermediate 14.3:

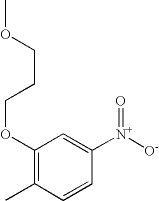

Intermediate 14.3 is synthesized by alkylation of 5-nitro-o-cresol (5.0 g, 32.6 mmol) analogously to the preparation of Intermediate 3.5. White amorphous material; ES-MS: M+H=226; HPLC: t_{Ret}=4.06 min.

Intermediate 15.1:

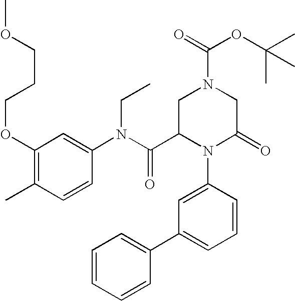

Intermediate 15.1 is synthesized by alkylation of Intermediate 14.1 (260 mg, 0.45 mmol) analogously to the preparation of Intermediate 5.3. White amorphous material; ES-MS: M+H=602; HPLC: t_{Ret}=5.02 min.

### EXAMPLE 16

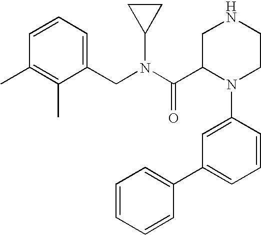

Example 16 is synthesized by deprotection of Intermediate 16.1 (132 mg, 0.25 mmol) analogously to the preparation of Example 1. White solid; ES-MS: M+H=440; HPLC: t_{Ret}=3.82 min.

Intermediate 16.1:

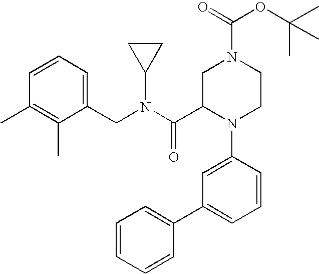

Intermediate 16.1 is synthesized by condensation of Intermediate 16.2 (120 mg, 0.31 mmol) analogously to the preparation of Intermediate 1.1. White amorphous material; ES-MS: M+H=540; HPLC: t_{Ret}=5.03 min.

Intermediate 16.2:

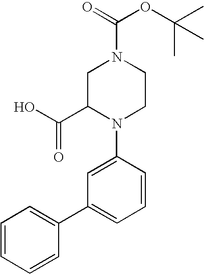

Intermediate 16.2 is synthesized by hydrolysis of Intermediate 16.3 (382 mg, 0.96 mmol) analogously to the preparation of Intermediate 1.2. White solid; ES-MS: M+H=383; HPLC: t_{Ret}=4.16 min.

Intermediate 16.3:

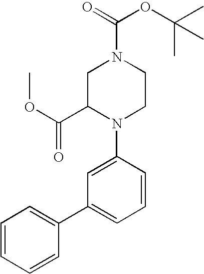

A mixture of Intermediate 1.3 (100 mg, 0.23 mmol) and 1M THF solution of BH₃-THF complex (1.38 mL, 1.38 mmol) in THF (3 mL) is stirred for 3 hours at room temperature. The reaction mixture is supplemented with H₂O and extracted with Et₂O. The combined organic phases are washed with H₂O and dried (Na₂SO₄). Concentration under reduced pressure and silica gel flash chromatography give Intermediate 16.3 as colorless oil; ES-MS: M+H=397, HPLC: t_{Ret}=4.75 min.

The following Examples enlisted on Table 2 are synthesized analogously to the preparation of Example 16. As far as not being commercially available, the synthesis of intermediates for the preparation of compounds of Examples 17-26 is described below the Table 2. The asterisk (*) marks the end of the bond via at which a moiety is bound to the rest of the molecule:

TABLE 2

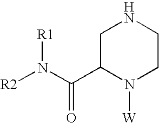

| Example | R1 | R2 | W | Analytical data |
|---|---|---|---|---|
| 17 | 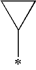 | 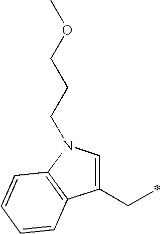 | 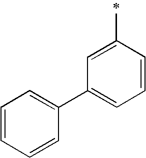 | MS: [M + 1]⁺ = 523HPLC t_{Ret} = 3.71 min. |
| 18 | 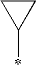 | 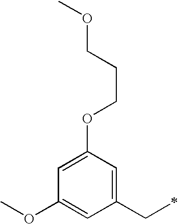 | 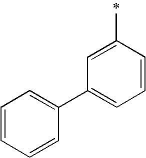 | MS: [M + 1]⁺ = 530HPLC t_{Ret} = 3.75 min. |
| 19 | 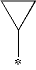 | 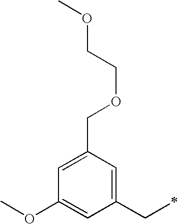 | 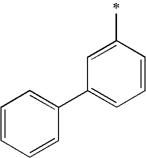 | MS: [M + 1]⁺ = 530HPLC t_{Ret} = 3.59 min. |
| 20 | 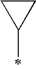 | 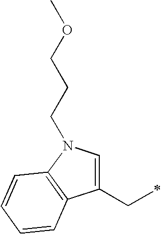 | 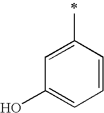 | MS: [M + 1]⁺ = 463HPLC t_{Ret} = 3.04 min. |
| 21 | 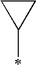 | 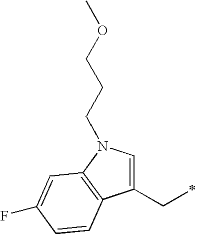 | 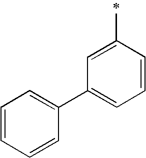 | MS: [M + 1]⁺ = 541HPLC t_{Ret} = 3.90 min. |
| 22 | 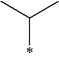 | 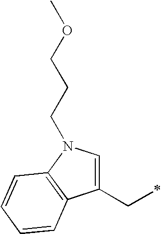 | 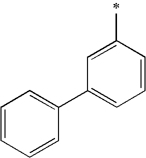 | MS: [M + 1]⁺ = 525HPLC t_{Ret} = 4.02 min. |
| 23 | 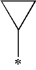 | 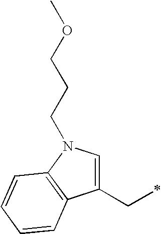 | 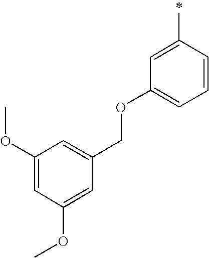 | MS: [M + 1]⁺ = 613HPLC t_{Ret} = 3.88 min. |
| 24 | 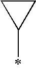 | 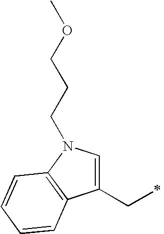 | 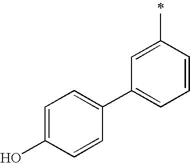 | MS: [M + 1]⁺ = 539HPLC t_{Ret} = 3.35 min. |
| 25 | 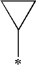 | 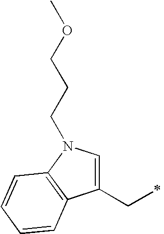 | 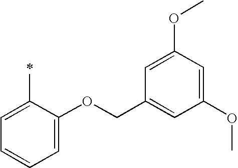 | MS: [M + 1]⁺ = 613HPLC t_{Ret} = 3.89 min. |
| 26 | 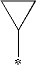 | 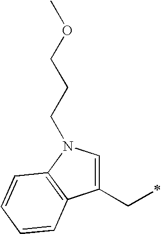 | 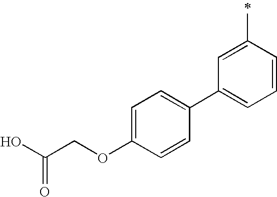 | MS: [M + 1]⁺ = 597HPLC t_{Ret} = 3.18 min. |

Intermediate 17.1:

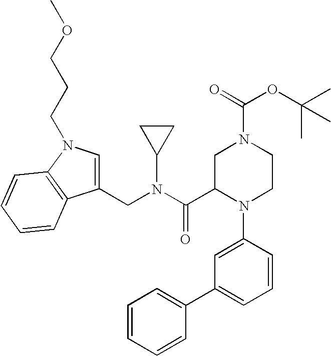

Intermediate 17.1 is synthesized by condensation of Intermediate 16.2 (120 mg, 0.31 mmol) and Intermediate 2.2 (106 mg, 0.41 mmol) analogously to the preparation of Intermediate 1.1. White amorphous material; ES-MS: M+H=623; HPLC: t_{Ret}=4.89 min.

Intermediate 18.1:

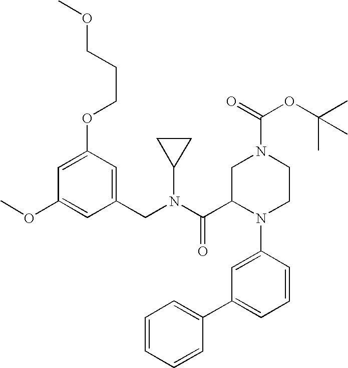

Intermediate 18.1 is synthesized by condensation of Intermediate 16.2 (200 mg, 0.52 mmol) and Intermediate 3.2 (143 mg, 0.68 mmol) analogously to the preparation of Intermediate 1.1. White solid; ES-MS: M+H=630; HPLC: t_{Ret}=4.99 min.

Intermediate 19.1:

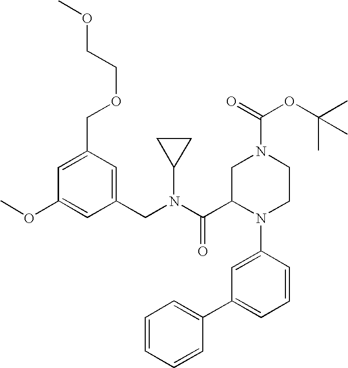

Intermediate 19.1 is synthesized by condensation of Intermediate 16.2 (205 mg, 0.54 mmol) and Intermediate 4.2 (185 mg, 0.70 mmol) analogously to the preparation of Intermediate 1.1. White solid; ES-MS: M+H=630; HPLC: t_{Ret}=4.78 min.

Intermediate 20.1:

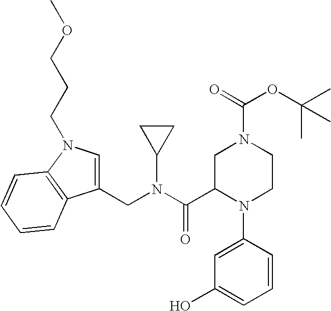

Intermediate 20.1 is synthesized by condensation of Intermediate 20.2 (300 mg, 1.24 mmol) and Intermediate 2.2 (417 mg, 1.60 mmol) analogously to the preparation of Intermediate 1.1. White amorphous material; ES-MS: M+H=563; HPLC: t_{Ret}=3.84 min.

Intermediate 20.2:

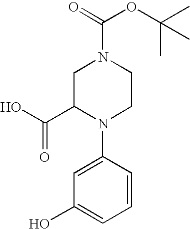

Intermediate 20.2 is synthesized by hydrolysis of Intermediate 20.3 (1.57 g, 4.4 mmol) analogously to the preparation of Intermediate 1.2. White solid; ES-MS: M+H=323; HPLC: t_{Ret}=3.00 min.

Intermediate 20.3:

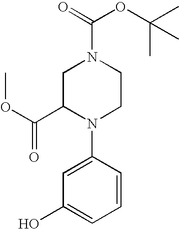

Intermediate 20.3 is synthesized by reduction of Intermediate 6.4 (2.0 g, 5.7 mmol) analogously to the preparation of Intermediate 16.3. White solid; ES-MS: M+H=337; HPLC: t_{Ret}=3.71 min.

Intermediate 21.1:

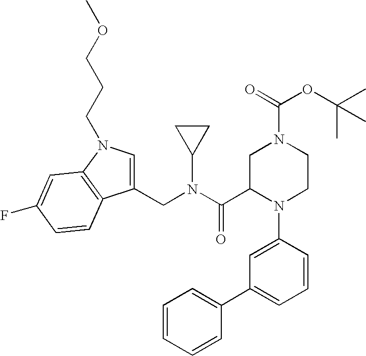

Intermediate 21.1 is synthesized by condensation of Intermediate 16.2 (197 mg, 0.55 mmol) and Intermediate 8.2 (149 mg, 0.71 mmol) analogously to the preparation of Intermediate 1.1. White solid; ES-MS: M+H=641; HPLC: t_{Ret}=5.07 min.

Intermediate 22.1:

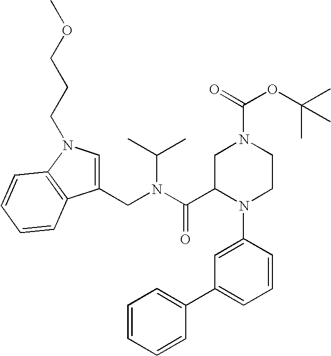

Intermediate 22.1 is synthesized by condensation of Intermediate 16.2 (150 mg, 0.39 mmol) and Intermediate 22.2 (152 mg, 0.59 mmol) analogously to the preparation of Intermediate 1.1. White solid; ES-MS: M+H=625; HPLC: t_{Ret}=5.19 min.

Intermediate 22.2:

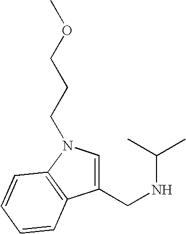

Intermediate 22.2 is synthesized by reductive amination of Intermediate 2.3 (3.3 g, 15 mmol) analogously to the preparation of Intermediate 2.2. Colorless oil; ES-MS: M+H=261; HPLC: t_{Ret}=2.74 min

Intermediate 23.1:

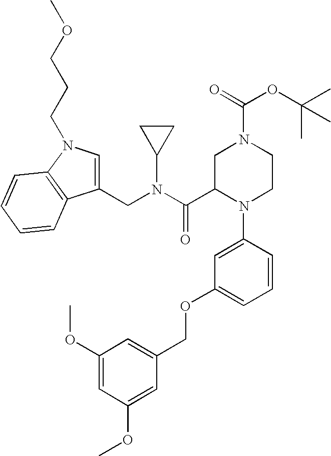

Intermediate 23.1 is synthesized by alkylation of Intermediate 20.1 (200 mg, 0.36 mmol) and 3,5-dimethoxybenzyl bromide (99 mg, 0.42 mmol) analogously to the preparation of Intermediate 2.3. White solid; ES-MS: M+H=713; HPLC: t_{Ret}=4.97 min.

Intermediate 24.1:

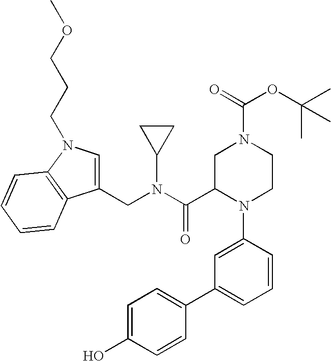

A mixture of Intermediate 24.2 (215 mg, 0.31 mmol), 4-hydroxyphenylboronic acid (64 mg, 0.47 mmol), K₃PO₄ (132 mg, 0.62 mmol) and Pd(PPh₃)₄ (35 mg, 0.03 mmol) in dioxane (5 mL) and H₂O (1 mL) is stirred at 60° C. for 2 h. After adding H₂O, the reaction mixture is extracted with EtOAc. The combined organic phases are washed with H₂O, brine and dried (MgSO₄). Concentration under reduced pressure and silica gel flash chromatography give Intermediate 24.1 as white amorphous material; ES-MS: M+H=639; HPLC: t_{Ret}=4.22 min.

Intermediate 24.2:

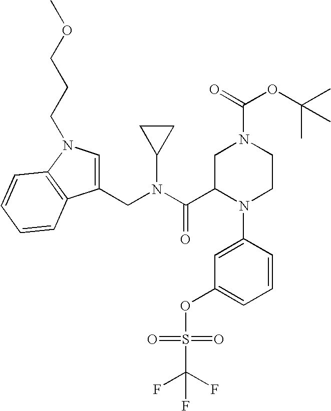

A mixture of Intermediate 20.1 (230 mg, 0.41 mmol), Tf₂O (83 μL, 0.49 mmol) and DIEA (0.18 mL, 1.0 mmol) in DCM (4 mL) is stirred at −78° C. for 30 min. After adding saturated NaHCO₃ solution, the reaction mixture is extracted with DCM. The combined organic phases are washed with H₂O, brine and dried (MgSO₄). Concentration under reduced pressure and silica gel flash chromatography give Intermediate 24.2. Colorless amorphous material; ES-MS: M+H=695; HPLC: t_{Ret}=5.46 min.

Intermediate 25.1:

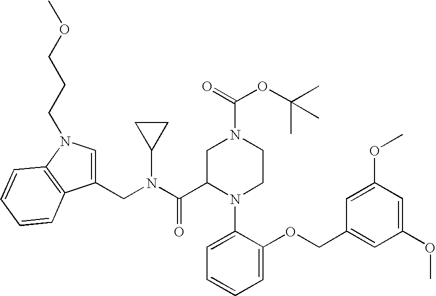

Intermediate 25.1 is synthesized by condensation of Intermediate 25.2 (155 mg, 0.33 mmol) and Intermediate 2.2 (111 mg, 0.43 mmol) analogously to the preparation of Intermediate 1.1. White solid; ES-MS: M+H=713; HPLC: t_{Ret}=4.60 min.

Intermediate 25.2:

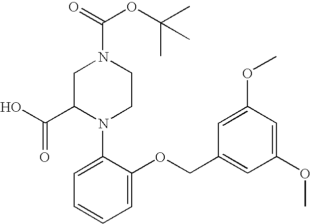

Intermediate 25.2 is synthesized by hydrolysis of Intermediate 25.3 (162 mg, 0.33 mmol) analogously to the preparation of Intermediate 1.2. White amorphous material; ES-MS: M+H=473; HPLC: t_{Ret}=4.26 min.

Intermediate 25.3:

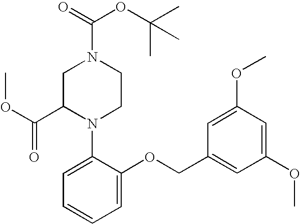

Intermediate 25.3 is synthesized by reduction of Intermediate 10.3 (280 mg, 0.56 mmol) analogously to the preparation of Intermediate 16.3. White solid; ES-MS: M+H=487; HPLC: t_{Ret}=4.90 min.

Intermediate 26.1:

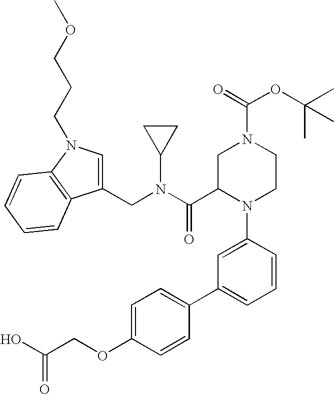

Intermediate 26.1 is synthesized by hydrolysis of Intermediate 26.2 (87.1 mg, 0.12 mmol) analogously to the preparation of intermediate 1.2. White amorphous material; ES-MS: M+H=697; HPLC: t_{Ret}=4.13 min.

Intermediate 26.2:

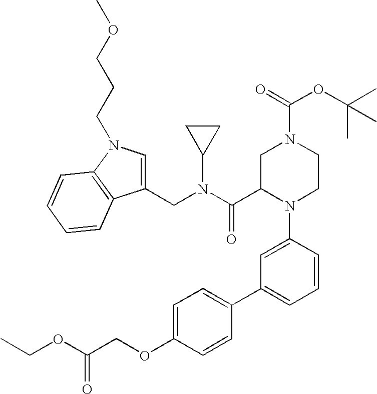

Intermediate 26.2 is synthesized by coupling of Intermediate 24.2 (400 mg, 0.58 mmol) and [4-(4,4,5,5-tetramethyl-1,3,2-dioxaborolan-2-yl)phenoxy]-acetic acid ethyl ester (265 mg, 0.86 mmol, WO2000027853) analogously to the preparation of Intermediate 24.1. White amorphous material; ES-MS: M+H=725; HPLC: t_{Ret}=4.78 min.

1. A compound of the formula I, 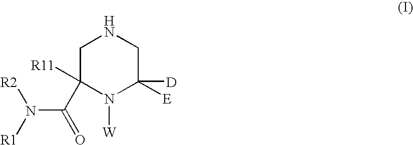
wherein
R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is a moiety selected from those of the formulae IA, IB and IC, 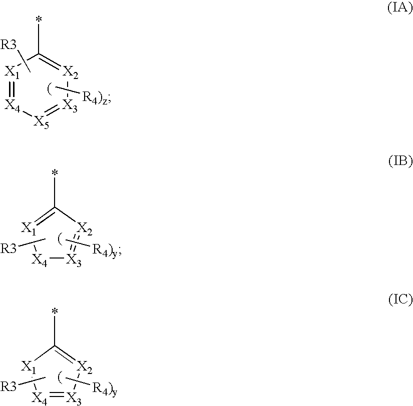
wherein the asterisk (*) denotes the position where the moiety W is bound to the 4-carbon in the piperidine ring in formula I, and wherein
X₁, X₂, X₃, X₄ and X₅ are independently selected from carbon and nitrogen, where X₄ in formula IB and X₁ in formula IC may have one of these meanings or further be selected from S and O, where carbon and nitrogen ring atoms can carry the required number of hydrogen or substituents R₃ or (if present within the limitations given below) R₄ to complete the number of bonds emerging from a ring carbon to four, from a ring nitrogen to three, with the proviso that in formula IA at least 2 of X₁ to X₅ are carbon and in formulae IB and IC at least one of X₁ to X₄ is carbon X₁ to X₄ are carbon;
y is 0, 1, 2 or 3;
z is 0, 1, 2, 3 or 4
R3 which can only be bound to any one of X₁, X₂, X₃ and X₄ is unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro or cyano;
R4 is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro and cyano;
each of D and E is hydrogen, or D and E together form oxo (═O); and
R11 is hydrogen, C₁-C₇-alkyl, halo-C₁-C₇-alkyl, cycloalkyl, halo-substituted cycloalkyl or cyano,
or a salt thereof.

2. (canceled)

3. A compound of the formula I according to claim 1 wherein
R1 is hydrogen, C₁-C₇-alkyl, C₃-C₈-cycloalkyl or C₃-C₈-cycloalkyl-C₁-C₇-alkyl;
R2 is phenyl, phenyl-C₁-C₇-alkyl, naphthyl, naphthyl-C₁-C₇-alkyl, indolyl, indolyl-C₁-C₇-alkyl, 2H-1,4-benzoxazin-3(4H)-onyl or 2H-1,4-benzoxazin-3(4H)-onyl-C₁-C₇-alkyl, wherein each phenyl, naphthyl, indolyl or 2H-1,4-benzoxazin-3(4H)-onyl is unsubstituted or substituted by one, two, or three moieties independently selected from C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy, C₁-C₇-alkoxy-C₁-C₇-alkoxy and halo;
W is a moiety of the formula IA wherein each of X₁, X₂, X₃, X₄, X₄ and X₅ is CH or a moiety of the formula IC wherein X₁, is S, X₂ is N, X₃ is CH and X₄ is CH, and R3 which is bound to any one of X₁, X₂, X₃,or X₄ in formula IA or to any one of X₃ and X₄ in formula IC is selected from the group consisting of phenyl, hydroxy, phenyloxy-C₁-C₇-alkyl and phenyl-C₁-C₇-alkoxy, where each phenyl mentioned in the present definition of W so far is unsubstituted or substituted by one or more moieties independently selected from hydroxy, C₁-C₇-alkoxy, carboxy-C₁-C₇-alkoxy, C₁-C₇-alkoxycarbonyl-C₁-C₇-alkoxy and phenyl- or naphthyl-C₁-C₇-alkoxycarbonyl-C₁-C₇-alkoxy;
each of y and z is 0;
each of D and E is hydrogen; or D and E together form oxo; and
R11 is hydrogen;
or a salt thereof.

4. A compound according to claim 1, having the formula 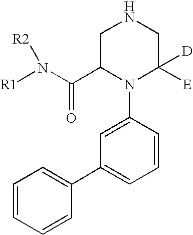
or a salt thereof.

5. A compound of the formula I according to claim 1, wherein
R1 is hydrogen, ethyl or cyclopropyl;
R2 is 3-(3-methoxypropoxy)-4-methoxy-phenyl, 3-(2-methoxyethyl)-4-methoxy-phenyl, 3-(3-methoxypropoxy)-4-methyl-phenyl, 3-(2-methoxyethyl)-4-methyl-phenyl, 2-(2,3-dimethylphenyl)-methyl, 3-(3-methoxy-propoxy-methyl)-5-methoxy-phenylmethyl, 3-(2-methoxy-ethoxy-methyl)-5-methoxy-phenylmethyl, 3-(3-methoxy-propoxy)-5-methoxy-phenylmethyl, 3-(2-methoxy-ethoxy)-5-methoxy-phenylmethyl, 1-(3-methoxy-propyl)-indol-3-yl-methyl, 1-2-methoxy-ethyl)-indol-3-yl-methyl, 5-fluoro-1-(3-methoxy-propyl)-indol-3-yl-methyl, 5-fluoro-1-(2-methoxy-ethyl)-indol-3-yl-methyl, 6-fluoro-1-(3-methoxy-propyl)-indol-3-yl-methyl, 6-fluoro-1-(2-methoxy-ethyl)-indol-3-yl-methyl, 4-(3-methoxypropyl)-2H-1,4-benzoxazin-3(4H)-on-6-yl or 4-(2-methoxyethyl)-2H-1,4-benzoxazin-3(4H)-on-6-yl,
W is 3-phenyl-phenyl, 3-hydroxyphenyl, 3-(4-hydroyphenyl)-phenyl, 3- or 2-[(3,5-dimethoxy-phenyl)-methoxy]-phenyl, 3-[(4-carboxyl-methyloxy)-phenyl]-phenyl or substituted or unsubstituted thiazolyl,
each of D and E is hydrogen or D and E together form oxo; and
R11 is hydrogen;
or a salt thereof.

6. A compound of the formula I according to claim 1, selected from the group of compounds having the following formulae: 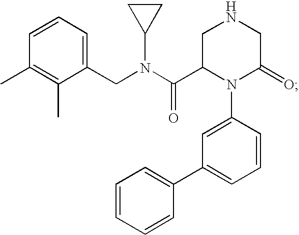 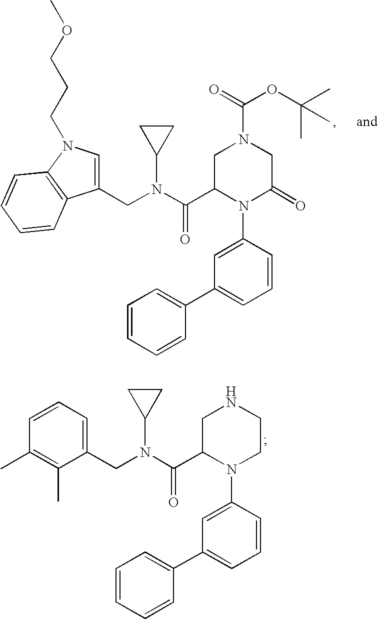
or a salt thereof.

7. A compound of the formula I according to claim 1, selected from the group of compounds of the formula I represented by the following formula and the definition of its moieties in the following table

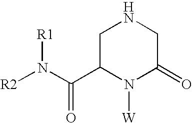

| Compound | R1 | R2 | W |
|---|---|---|---|
| 3 | 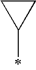 | 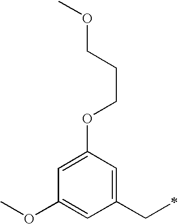 | 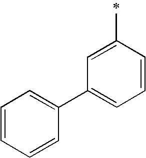 |
| 4 | 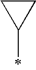 | 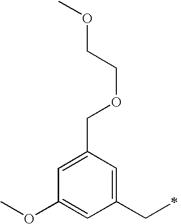 | 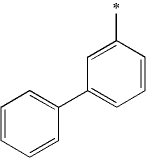 |
| 5 | 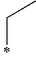 | 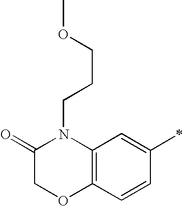 | 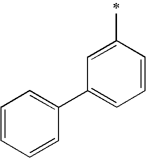 |
| 6 | 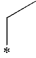 | 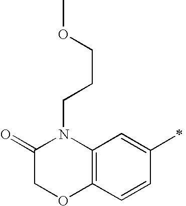 | 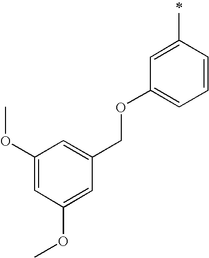 |
| 7 | 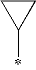 | 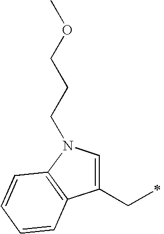 | 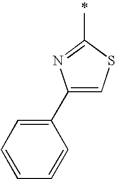 |
| 8 | 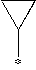 | 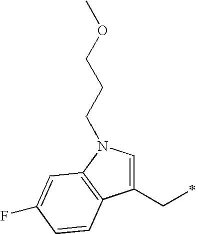 | 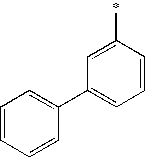 |
| 9 | 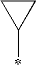 | 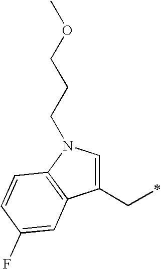 | 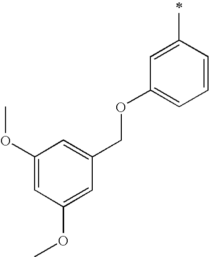 |
| 10 | 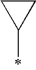 | 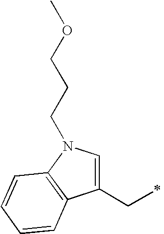 | 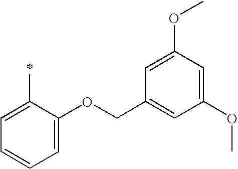 |
| 11 | 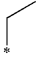 | 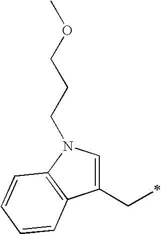 | 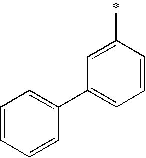 |
| 12 | H | 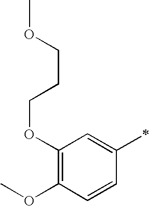 | 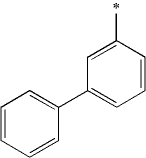 |
| 13 | 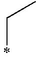 | 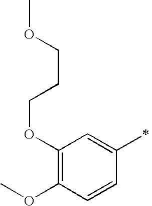 | 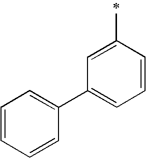 |
| 14 | H | 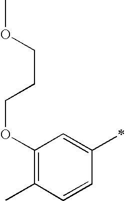 | 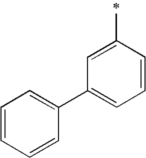 |
| 15 | 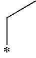 | 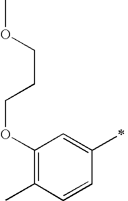 | 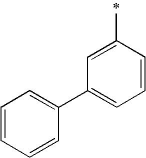 |

8. A compound of the formula I according to claim 1, selected from the group of compounds of the formula I represented by the following formula and the definition of its moieties in the following table

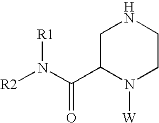

| Compound | R1 | R2 | W |
|---|---|---|---|
| 17 | 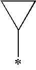 | 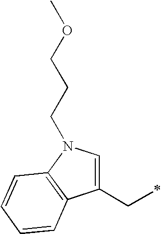 | 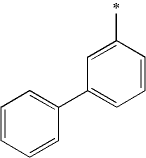 |
| 18 | 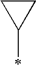 | 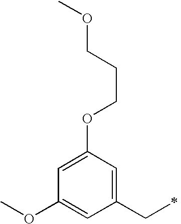 | 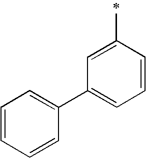 |
| 19 | 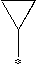 | 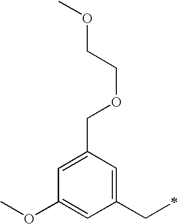 | 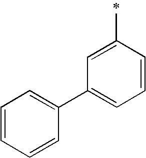 |
| 20 | 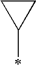 | 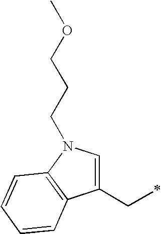 | 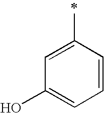 |
| 21 | 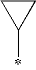 | 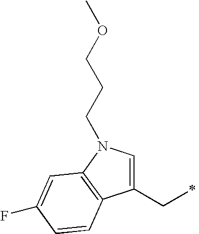 | 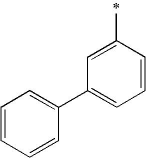 |
| 22 | 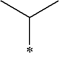 | 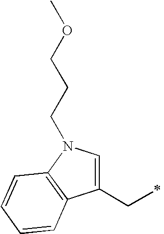 | 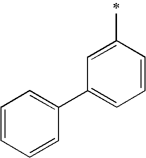 |
| 23 | 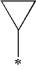 | 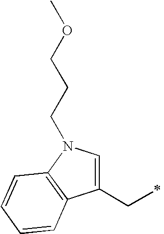 | 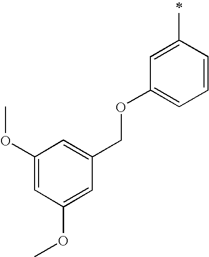 |
| 24 | 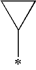 | 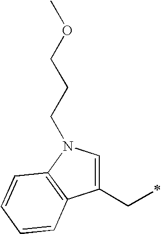 | 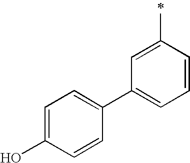 |
| 25 | 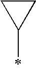 | 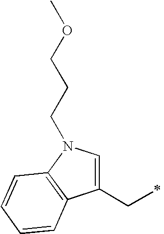 | 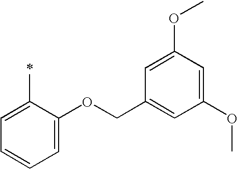 |
| 26 | 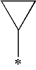 | 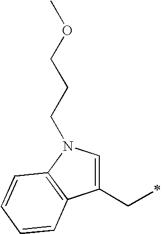 | 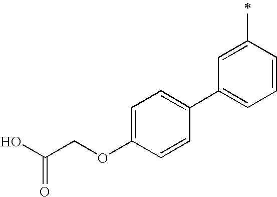 |

9. A compound of the formula I according to claim 1,
with the configuration shown in the following formula (A), 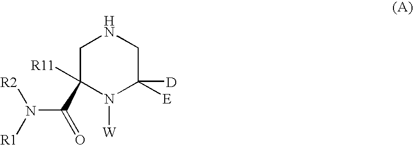
or with the configuration shown in the following formula (B) 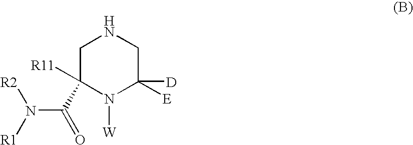
or a salt thereof.

10-13. (canceled)

14. A pharmaceutical formulation, comprising a compound of claim 1, or a pharmaceutically acceptable salt thereof, and at least one pharmaceutically acceptable carrier material.

15. A method of treatment a disease that depends on activity of renin, comprising administering to a warm-blooded animal, in need of such treatment a pharmaceutically effective amount of a compound of claim 1, or a pharmaceutically acceptable salt thereof.

16. A process for the manufacture of a compound of claim 1, or a pharmaceutically acceptable salt thereof, said process comprising
reacting a carbonic acid of the formula II, 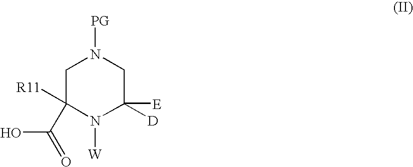
or a reactive derivative thereof, wherein PG is a protecting group and W and R11 are as defined in claim 1, with an amino compound of the formula III,
R1-NH—R2 (III)
wherein R1 and R2 are as defined in claim 1.
